SEQUENCE LISTING (4 sequences)

SEQ ID NO: 1 - DNA, 20 bp, synthetic construct
  source 1..20: mol_type = other DNA
aatctcccaccgtcagtacg

SEQ ID NO: 2 - DNA, 24 bp, synthetic construct
  source 1..24: mol_type = other DNA
cagcacctacatcttgggtggaga

SEQ ID NO: 3 - DNA, 24 bp, synthetic construct
  source 1..24: mol_type = other DNA
actgttcttcctcggagccttgtc

SEQ ID NO: 4 - DNA, 21 bp, synthetic construct
  source 1..21: mol_type = other DNA
cgttttctgagcatacctgga

(19) United States
(12) Patent Application Publication
(10) Pub. No.: US 2024/0285631 A1
(43) Pub. Date: Aug. 29, 2024

(54) METHODS OF TREATING VASCULAR MALFORMATIONS

(71) Applicants: Karen K. Hirschi, Charlottesville, VA (US); Gael Genet, Charlottesville, VA (US); Shelby Rose Cain, Charlottesville, VA (US); Nafiisha Genet, Charlottesville, VA (US)

(72) Inventors: Karen K. Hirschi, Charlottesville, VA (US); Gael Genet, Charlottesville, VA (US); Shelby Rose Cain, Charlottesville, VA (US); Nafiisha Genet, Charlottesville, VA (US)

(21) Appl. No.: 18/428,746

(22) Filed: Jan. 31, 2024

Related U.S. Application Data
(60) Provisional application No. 63/482,444, filed on Jan. 31, 2023.

(51) Int. Cl. A61K 31/519; A61P 9/14

(52) U.S. Cl. CPC A61K 31/519; A61P 9/14

(57) ABSTRACT

The present disclosure provides for methods of treating vascular malformation using cell cycle inhibitors, such as palbociclib, ribociclib, CVT-313, and abemaciclib, as well as pharmaceutical compositions. The methods can include treating adults that have developed a vascular malformation, treating neonates that may develop a vascular malformation, or treating children that have developed a vascular malformation or that may develop a vascular malformation.

### CROSS-REFERENCE TO RELATED APPLICATION

This application claims priority to U.S. provisional application entitled “INDUCED ENDOTHELIAL CELL CYCLE ARREST PREVENTS ARTERIO-VENOUS MALFORMATIONS IN HEREDITARY HEMORRHAGIC TELANGIECTASIA” having Ser. No. 63/482,444 filed on Jan. 31, 2023, which is entirely incorporated herein by reference.

### STATEMENT ON FUNDING PROVIDED BY THE U.S. GOVERNMENT

This invention was made with Government support under contract HL146056 awarded by the National Institutes of Health. The Government has certain rights in the invention.

### SEQUENCE LISTING

This application contains a sequence listing filed in ST.26 format entitled “222117-1130 Sequence Listing” created on Jan. 30, 2024, having 4,389 bytes. The content of the sequence listing is incorporated herein in its entirety.

### BACKGROUND

Distinct endothelial cell cycle states (early G1 vs. late G1) provide different “windows of opportunity” to enable the differential expression of genes that regulate venous vs. arterial specification, respectively. Endothelial cell cycle control and arterial-venous identities are disrupted in vascular malformations including arteriovenous (AV) shunts, the hallmark of hereditary hemorrhagic telangiectasia (HHT). To date, the mechanistic link between endothelial cell cycle regulation and the development of AV malformations (AVMs) in HHT is not known.

### SUMMARY

The present disclosure provides for methods of treating vascular malformation using cell cycle inhibitors, such as palbociclib, ribociclib, CVT-313, and abemaciclib. The methods can include treating adults that have developed a vascular malformation, treating neonates that may develop a vascular malformation, or treating children that have developed a vascular malformation or that may develop a vascular malformation.

The present disclosure provides for methods for treating a vascular malformation, comprising: administering to a subject in need thereof, a pharmaceutical composition, wherein the pharmaceutical composition comprises a therapeutically effective amount of the pharmaceutical composition, wherein the pharmaceutical composition includes: palbociclib or pharmaceutically acceptable salts thereof; ribociclib or pharmaceutically acceptable salts thereof, CVT-313 or pharmaceutically acceptable salts thereof; or abemaciclib, or pharmaceutically acceptable salts thereof. The subject can be an adult or a child or a neonate.

The present disclosure provides for methods to prevent or treat endothelial cell hyperproliferation comprising administering to a subject in need thereof a pharmaceutical composition, wherein the pharmaceutical composition comprises a therapeutically effective amount of the pharmaceutical composition, wherein the pharmaceutical composition includes: palbociclib or pharmaceutically acceptable salts thereof; a ribociclib or pharmaceutically acceptable salts thereof; a CVT-313 or pharmaceutically acceptable salts thereof; or abemaciclib, or pharmaceutically acceptable salts thereof.

In a particular aspect, the pharmaceutical composition can include a therapeutically effective amount of a compound to treat or prophylactically treat a vascular malformation in an adult, child, or neonate. The pharmaceutical composition includes: palbociclib or pharmaceutically acceptable salts thereof, ribociclib or pharmaceutically acceptable salts thereof; CVT-313 or pharmaceutically acceptable salts thereof; or abemaciclib, or pharmaceutically acceptable salts thereof.

### BRIEF DESCRIPTION OF DRAWINGS

Further aspects of the present disclosure will be more readily appreciated upon review of the detailed description of its various embodiments, described below, when taken in conjunction with the accompanying drawings.

FIGS. 1A-1E describe endothelial cell gene expression and cell cycle state in HHT. FIG. 1A is a schematic of the Fucci2 reporter where cells in early G1 are reporter negative, late G1 cell nuclei are red and S/G2/M cell nuclei are green. FIG. 1B illustrates a timeline used for treatment with BMP9/10 blocking antibodies (BMP9/10 Abs) in Fucci2 postnatal mice. FIG. 1C illustrates representative confocal images showing flat-mounted retinas, with ECs labelled with anti-ERG and IB4, from either control or BMP9/10 Abs-treated Fucci2 postnatal day (P)7 pups. Bar graphs are quantifications of ECs (%) in early G1, late G1 and S/G2/M phases in arteries, veins, and capillaries in control vs. BMP9/10 Abs-treated animals (N=6 retinas per group). Data are mean±SEM, ***p≤0.001, **p≤0.01, *p≤0.05, ns p>0.05. Two-Way ANOVA for independent measures was used. p<0.05 was considered statistically significant. White arrow heads: AV shunts. V: Vein, A: Artery. FIG. 1D illustrates Z-score heat map showing the differential expression of genes regulating cell cycle progression in primary retinal mouse ECs of control vs. BMP9/10 Abs-treated pups (N=3 different experiments/group). FIG. 1E illustrates Ki67 and Hematoxylin and Eosin (H&E) staining of biopsies of healthy skin in resection borders from patients with melanoma (Patients A and B) and skin telangiectases from patients with HHT type 2 (Patients C and D). Right panel shows quantifications of Ki67⁺-ECs (N=3 patients/group. Data are mean±SEM, Mann-Whitney U test). L: vessel lumen. Red arrows point to Ki67⁺ ECs. Scale bar in (A): 200 mm, (E): 20 mm.

FIGS. 2A-2I describe palbociclib as preventing AVMs induced by BMP9/10 blocking antibodies. FIG. 2A illustrates a timeline used for dosing of C57BL/6 pups treated with anti-BMP9/10 Abs and CDK4/6i. FIG. 2B illustrates representative images showing retinal vasculature, with ECs labelled with IB4, from control, BMP9/10 Abs-treated or BMP9/10 Abs- and CDK4/6i-treated P7 animals. Red arrow heads indicate AVMs, and Red * show hyperproliferative plexi. FIGS. 2C-2G illustrate bar graphs showing quantifications of shunt number per retina (FIG. 2C), vascular density (FIG. 2D), vascular progression (FIG. 2E) (d: vascular coverage length; D: retinal petal length), and venous (FIG. 2F) and arterial (FIG. 2G) vessel diameter. (N=8 to 13 retinas/group, at least 4 mice). Data are mean±SEM, One-Way ANOVA with Holm-Šidàk's multiple comparison test: **p≤0.01, *p≤0.05, ns p>0.05. FIG. 2H illustrates retinal tissue flat mounts with anti-ERG staining (white) of ECs in early G1 (reporter negative), late G1 (red) and S/G2/M (green) in venous, arterial and capillary vessels of control, BMP9/10 Abs-treated, and BMP9/10 Abs- and CDK4/6i-treated P7 Fucci2 animals. FIG. 2I illustrates quantifications of the percentage of ECs in early G1, late G1 and S/G2/M phases in veins, arteries and capillaries of control, BMP9/10 Abs-treated and BMP9/10 Abs- and CDK4/6i-treated P7 Fucci2 animals (N=6 retinas/group). Data are mean±SEM, ***p≤0.001, **p≤0.01, *p≤0.05. Two-Way ANOVA for independent measures was used. p<0.05 was considered statistically significant. FIG. 2J illustrates a Z-score heat map showing the differential expression of genes regulating cell cycle progression from G1/S to M phases in primary retinal mouse ECs of BMP9/10 Abs-treated vs. BMP9/10 Abs- and CDK4/6i-treated pups (N=3 different bulk RNAseq experiments/group). Scale bars (B): 400 mm, (H): 50 mm.

FIGS. 3A-3F describe induced-late G1 cell cycle arrest promotes endothelial cell polarized migration. FIG. 3A illustrates gene ontology (GO) term analysis of selected gene families modified in primary retinal mouse ECs of BMP9/10 Abs- and CDK4/6i-treated vs BMP9/10 Abs-treated pups (N=3 different experiments/group). FIG. 3B illustrates Z-score heat map showing the differential expression of genes regulating cell migration in primary retinal mouse ECs of BMP9/10 Abs-treated vs BMP9/10 Abs- and CDK4/6i-treated pups (N=3 different experiments/group). FIG. 3C illustrates the polarity axis of each cell was defined as the angle between the direction of blood flow and the cell polarity axis, defined by a vector drawn from the center of the cell nucleus to the center of the Golgi apparatus. FIG. 3D illustrates EC polarization in arteries and veins of control, BMP9/10 Abs-treated, and BMP9/10 Abs- and CDK4/6i-treated animals. EC nuclei are immunostained with anti-ERG1,2,3 and Golgi apparatus with anti-GM130; ECs are labelled with IB4. (Yellow arrows: direction of blood flow; green head arrows indicate EC Golgi orientation towards blood flow direction. A: Artery; V: Vein). FIG. 3E illustrates angular histograms showing the distribution of polarization angles of retinal ECs in arteries and veins from control, BMP9/10 Abs-treated, and BMP9/10 Abs- and CDK4/6i-treated P6 animals (N=6 to 8 retinas). FIG. 3F illustrates polarity index dot plots of ECs from arteries (A) and veins (V) from control, BMP9/10 Abs-treated, and BMP9/10 Abs- and CDK4/6i-treated P6 animals (N=6 to 8 retinas. Data are mean±SEM **p≤0.01, ns p>0.05). Two-Way ANOVA for independent measures was used. p≤0.05 was considered statistically significant. Scale bar (D): 200 mm.

FIGS. 4A-4E describe induced-late G1 cell cycle arrest prevents pathological metabolic rewiring in ECs. Z-score heat map showing the differential expression of genes regulating metabolic pathways in primary retinal mouse ECs of control vs. BMP9/10 Abs-treated (FIG. 4A) and BMP9/10 Abs-treated vs BMP9/10 Abs- and CDK4/6i-treated (FIG. 4B) pups (N=3 different experiments/group). FIG. 4C illustrates representative traces of oxygen consumption rate (OCR) measured by Seahorse assay of control vs. CDK4/6i-treated HUVECs in response to oligomycin (ATP synthase inhibitor), BAM15 (mitochondrial uncoupler) and Antimycin A/Rotenone (mitochondrial respiratory chain complex III and I inhibitors) (N=6 independent experiments, Data are mean±SEM **p≤0.01, ns p>0.05). Two-way ANOVA for independent measures with Bonferonni's correction for statistical analysis. p<0.05 was considered statistically significant. The normality and homogeneity of variances assumptions were tested using Shapiro-Wilk test (p>0.05) and Mauchly's test of sphericity respectively. Quantification of mitochondrial activity expressed as basal OCR (FIG. 4D) and respiratory capacity (FIG. 4E) in control vs. CDK4/6i-treated HUVECs. (N=6 independent experiments, Data are mean±SEM Mann-Whitney U test: ***p<0.001, **p≤0.01,*p<0.05, ns p>0.05).

FIGS. 5A-5I describe palbociclib prevents vascular malformations in HHT type 2. FIG. 5A illustrates a timeline used for tamoxifen (Tx) injection and CDK4/6i treatment of Alk1EC^{iKO} pups. FIG. 5B illustrates representative images showing retinal vasculature labelled with IB4 from control, Alk1EC^{iKO}, and Alk1EC^{iKO}+CDK4/6i-treated P5 animals. Bar graphs showing (FIG. 5C) quantification of AV shunt number per retina, (FIG. 5D) vascular density, and (FIG. 5E) vascular progression. (N=8 to 10 retinas/group, at least 4 mice). Data are mean±SEM, One-way ANOVA, *p≤0.05, **p≤0.01). FIG. 5F illustrates a timeline used for Tx injection and CDK4/6i treatment of Alk1EC^{iKO} pups for blue latex dye perfusion experiments. FIG. 5G illustrates blue latex dye vascular perfusion in brains from control, Alk1EC^{iKO}, and Alk1EC^{iKO}+CDK4/6i-treated P7 animals. Red arrows show latex filled veins indicating the presence of AVMs. FIG. 5H illustrates the quantification of left middle cerebral artery (MCA) and basilar artery (BA) in control, Alk1EC^{iKO}, and Alk1EC^{iKO}+CDK4/6i-treated animals. FIG. 5I illustrates the quantification of the number of latex filled veins in control, Alk1EC^{iKO}, and Alk1EC^{iKO}+CDK4/6i-treated animals. (N=4 to 6 mice/group. Data are mean±SEM, one-way ANOVA with Holm-Šidàk's multiple comparison test, *p≤0.05, **p≤0.01, ***p<0.001). Scale bar (G): 4 mm.

FIGS. 6A-6H describe single cell RNA sequencing analysis of arteriovenous identity in ECs isolated from Alk1EC^{iKO}, with or without Palbociclib treatment. FIG. 6A illustrates the timeline used for tamoxifen (Tx) injection and CDK4/6i treatment of Alk1EC^{iKO} pups. FIG. 6B illustrates a schematic of protocol for single cell isolation and single cell RNA sequencing (scRNAseq) of ECs isolated from P5 Alk1^{fl/fl}, Alk1^{fl/fl} treated with CDK4/6i, Alk1EC^{iKO}, and Alk1EC^{iKO} treated with CDK4/6i. FIG. 6C illustrates PHATE dimensionality reduction plot with clusters of EC subtypes. FIG. 6D illustrates dot plot of cell type-specific gene expression in the EC clusters from (FIG. 6C). FIG. 6E illustrates the arterial score of individual cells in PHATE dimensionality reduction plot in the indicated groups. FIG. 6F illustrates dot-plot analysis displaying the expression of specific genes known to be enriched in arterial ECs in the indicated groups. FIG. 6G illustrates the venous score of individual cells in PHATE dimensionality reduction plot in the indicated groups. FIG. 6H illustrates a dot-plot analysis displaying the expression of specific genes known to be enriched in venous ECs in the indicated groups.

FIGS. 7A-E describe cell cycle state analysis of Alk1EC^{iKO} animals treated with palbociclib. FIG. 7A illustrates a timeline used for tamoxifen (Tx) injection and CDK4/6i treatment of Alk1EC^{iKO} pups. FIG. 7B illustrates quantifications of ECs (%) in early G1, late G1 and S/G2/M phases in veins, arteries, and capillaries of indicated groups (N=6 retinas/group. Data are mean±SEM, *p≤0.05. Two-Way ANOVA for independent samples was used. p≤0.05 was considered statistically significant. FIG. 7C illustrate retinal tissue flat mounts with anti-ERG staining of ECs (white) in early G1 (reporter negative), late G1 (red) and S/G2/M (green) in arterial, venous, and capillary vessels of P5 Alk1^{fl/fl}, Alk1^{fl/fl} treated with CDK4/6i, Alk1EC^{iKO}, and Alk1EC^{iKO} treated with CDK4/6i animals. Insets shows IB4 staining to visualize vasculature organization. V: vein, A: artery. FIG. 7D illustrates late G1 score of individual cells in PHATE dimensionality reduction plot in the indicated groups. FIG. 7E illustrates early G1 score of individual cells in PHATE dimensionality reduction plot in the indicated groups. Scale bars (FIG. 7C): 100 mm.

FIGS. 8A-8H describes palbociclib promotes BMP9 signaling and inhibits VEGF signaling in endothelial cells. FIG. 8A illustrates Western Blot analysis of the phosphorylation of the indicated proteins in response to VEGF-A (1.5 nM) in control vs. CDK4/6i treated HUVECs. FIG. 8B illustrates Western Blot analysis of the phosphorylation of the indicated proteins in response to BMP9 (10 ng/ml) in control vs. CDK4/6i-treated HUVECs. FIG. 8C illustrates quantification of phosphorylation normalized to total protein levels. (N=5 independent experiments, Data are mean±SEM, Mann-Whitney U test: *p<0.05). FIG. 8D illustrates Z-score heat map showing the differential expression of genes that regulate BMP9/10 and VEGF signaling in primary retinal mouse ECs of BMP9/10 Abs-treated vs. BMP9/10 Abs- and CDK4/6i-treated pups (N=3 different bulk RNAseq experiments/group). FIG. 8E illustrates ENDOGLIN (green) and IB4 (white) staining of retinal flat-mounts from P5 Alk1^{fl/fl}, Alk1^{fl/fl} treated with CDK4/6i, Alk1EC^{iKO}, and Alk1EC^{iKO} treated with CDK4/6i animals (A: Artery; V: Vein). FIG. 8F illustrates quantification of fluorescence intensity of ENDOGLIN staining in (FIG. 8E) retinal venous vessels of the indicated animals (N=4 retinas per group. Data are mean±SEM, one-way ANOVA with Tukey's multiple comparison test: *p<0.05; ns p>0.05). FIG. 8G illustrates timeline used for Tx injection, and CDK4/6i and FLT1 blocking antibody treatment of Alk1EC^{iKO} pups. Representative images showing retinal vasculature labelled with IB4 from control, Alk1EC^{iKO}, Alk1EC^{iKO}+CDK4/6i, and Alk1EC^{iKO}+CDK4/6i-+FLT1 Ab-treated P5 animals (A: artery; V:

vein; Red arrow heads indicate AV shunts). FIG. 8H illustrates quantification of AV shunt number per retina (N=6 retinas/group). Data are mean±SEM, ***p≤0.001, **p≤0.01. Two-Way ANOVA for independent samples was used. p<0.05 was considered statistically significant. Scale (E): 100 mm, (G): 200 mm.

FIG. 9A illustrates the timeline used for treatment with BMP9/10 Abs and EdU in C57BL/6 pups. FIG. 9B illustrates representative images showing retinal vasculature labelled with IB4 and EdU from control and BMP9/10 Abs-treated P7 animals. A: artery; V: vein. Dashed squares indicate the vascular regions analyzed. FIG. 9C illustrates the quantification of EdU⁺ and IB4⁺ cells per retinal vessel length (mm) from control and BMP9/10 Abs-treated animals. FIG. 9D illustrates the quantification of EdU⁺ and IB4⁺ cells in depicted retinal capillaries and plexi area above arteries and veins (mm²). (N=5 to 7 retinas/group. Data are mean±SEM, Mann-Whitney Test, *p<0.05). FIG. 9E illustrates the Gene Ontology (GO) term analysis of selected gene families modified in primary retinal mouse ECs of BMP9/10 Abs-treated vs control pups (N=3 different experiments/group). Scale bar (B): 200 mm.

FIGS. 10A and 10B illustrate the timeline used for CDK4/6i treatment alone in C57BL/6 pups and representative images showing retinal vasculature of P7 animals labelled with IB4 (N=3 to 4 animals/group). Vascular perfusion assessed using blue latex dye in lungs (FIGS. 10C-D), duodenum (FIGS. 10E-F) and brain (FIGS. 10G-H) from control, BMP9/10 Abs-treated, BMP9/10 Abs- and CDK4/6i-treated P7 animals. Lung samples have been clarified. Quantifications of intrapulmonary artery diameter (FIG. 10D) and intestinal capillary diameter (FIG. 10F). Red arrows indicate intestinal capillaries (N=3 to 7 animals/group. Data are mean±SEM, one-way ANOVA with Holm-Šidàk's multiple comparison test, **p≤0.01, *p≤0.05, ns p>0.05). Quantification of left middle cerebral artery (MCA) and basilar artery (BA) diameter (FIG. 10H). (N=5 to 9 animals/group. Data are mean±SEM, one-way ANOVA with Holm-Sidak's multiple comparison test, ns p>0.05). Scale bars: (FIGS. 10C and 10G): 2 mm, (E): 1 mm.

FIG. 11A illustrates quantifications of ECs (%) in early G1, late G1 and S/G2/M states in the retinal distal plexus of BMP9/10 Abs-treated and BMP9/10 Abs- and CDK4/6i-treated P7 Fucci2 animals (N=6 retinas/group. Data are mean±SEM, Two-way ANOVA for repeated measures with Bonferonni's correction for statistical analysis, ***p≤0.001, *p≤ 0.05, ns p>0.05). FIG. 11B illustrates Western Blot analysis of Rb Ser 807/811 phosphorylation in response to Palbociclib (CDK4/6i) (24 h) in HUVECs. Quantification of Rb phosphorylation normalized to bactin (N=5 independent experiments. Data are mean±SEM, Mann-Whitney U test: **p≤0.01). FIG. 11C illustrates the working model of Palbociclib effects on endothelial cell cycle regulation in BMP9 signaling deficiency.

FIG. 12A illustrates the timeline used for BMP9/10 Abs and CDK4/6i treatments in C57bl/6 pups. FIGS. 12B-F JAGGED1 (green) and IB4 (white) in (FIG. 12B), aSMA (green) and IB4 (white) staining in (FIG. 12D) and ENDOMUCIN (green) and IB4 (white) in (FIG. 12F) of retinal flat-mounts from control, BMP9/10 Abs-treated, BMP9/10 Abs- and CDK4/6i-treated P7 C57bl/6 animals. (A: artery; V: vein; white arrow heads: AVMs). Quantification of JAGGED1 fluorescence intensity in retinal arterial ECs (FIG. 12C), of aSMA arterial coverage (FIG. 12E) and ENDOMUCIN in retinal arteries (FIG. 12G) (N=4 animals/group, 10 to 14 region of interest analyzed/group. Data are mean±SEM, one-way ANOVA with Holm-Sidak's multiple comparison test, ns p>0.05; ***p<0.01). Scale bars: 200 mm.

FIGS. 13A-C illustrate representative images of blue latex dye vascular perfusion in lungs (FIG. 13A) duodenum capillaries (FIG. 13B) and brains (dorsal view) (FIG. 13C) from Alk1^{fl/fl} (N=4 mice), Alk1^{ECiKO} (N=7 mice) and Alk1^{ECiKO}+CDK4/6i (N=6 mice) treated P7 animals. A: Artery; V: Vein. Scale bars: 2 mm (FIGS. 13A and C), 1 mm (FIG. 13B).

FIG. 14A illustrates a dot-plot analysis displaying expression of genes involved in the TGFb signaling pathway in the arterial EC cluster in the indicated groups. FIG. 14B illustrates dot-plot analysis displaying expression of genes involved in the BMP signaling pathway in the venous EC cluster in the indicated groups.

FIG. 14A is a dot-plot analysis displaying expression of genes involved in the TGFβ signaling pathway in the arterial EC cluster in the indicated groups. FIG. 14B is a dot-plot analysis displaying expression of genes involved in the BMP signaling pathway in the venous EC cluster in the indicated groups.

FIG. 15A illustrates Western Blot analysis of the AKT phosphorylation in response to VEGF-A (1.5 nM) in Alk1 siRNA-silenced HUVECs treated, or not, with CDK4/6i. FIG. 15B illustrates quantification of AKT phosphorylation normalized to total AKT protein levels. (N=8 independent experiments, Data are mean±SEM, Mann-Whitney U test: ns p<0.05). FIG. 15C illustrates a dot-plot analysis displaying expression of genes involved in the AKT signaling pathway in all EC clusters in the indicated groups.

FIG. 16 illustrates palbociclib, CVT-313 and ribociclib dose response effects on proliferation of human umbilical vein endothelial cells assessed by EdU uptake.

FIG. 17 illustrates, at adult stages, Alk1 mutant animals (HHT model) present abnormal brain vasculature 1 week after induction of gene deletion by tamoxifen. Euthanized animals have been perfused with colored reagent to visualize the blood vessels.

FIG. 18 illustrates, at adult stages, Alk1 mutant animals (HHT model) present severe abnormal and dense brain vasculature 3 weeks after induction of gene deletion by tamoxifen. However, Alk1 animals that received 3 consecutive doses of Palbociclib (CDK4/6i) (120 mg/kg, oral gavage) at day 7, 9 and 11 after tamoxifen injection have brain vasculature similar to control animals. This result demonstrates that Palbociclib regressed brain vascular anomalies in adult Alk1 mutant animals.

FIG. 19 illustrates the structure for palbociclib, ribociclib, CVT-313, and abemaciclib.

### DETAILED DESCRIPTION

This disclosure is not limited to particular embodiments described, and as such may, of course, vary. The terminology used herein serves the purpose of describing particular embodiments only, and is not intended to be limiting, since the scope of the present disclosure will be limited only by the appended claims.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, dimensions, frequency ranges, applications, or the like, as such can vary. It is also to be understood that the terminology used herein is for purposes of describing particular embodiments only, and is not intended to be limiting. It is also possible in the present disclosure that steps can be executed in different sequence, where this is logically possible. It is also possible that the embodiments of the present disclosure can be applied to additional embodiments involving measurements beyond the examples described herein, which are not intended to be limiting. It is furthermore possible that the embodiments of the present disclosure can be combined or integrated with other measurement techniques beyond the examples described herein, which are not intended to be limiting.

It should be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Prior to describing the various embodiments, the following definitions are provided and should be used unless otherwise indicated.

### Definitions

It is understood that “substitution” or “substituted” includes the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc.

The term “composition” as used herein refers to a product comprising the specified ingredients in the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure and a pharmaceutically acceptable carrier.

When a compound of the present disclosure is used contemporaneously with one or more other drugs, a pharmaceutical composition containing such other drugs in addition to the compound of the present disclosure is contemplated. Accordingly, the pharmaceutical compositions of the present disclosure include those that also contain one or more other active ingredients, in addition to a compound of the present disclosure. The weight ratio of the compound of the present disclosure to the second active ingredient may be varied and will depend upon the effective dose of each ingredient. Generally, an effective dose of each will be used. Thus, for example, but not intended to be limiting, when a compound of the present disclosure is combined with another agent, the weight ratio of the compound of the present disclosure to the other agent will generally range from about 1000:1 to about 1:1000, preferably about 200:1 to about 1:200. Combinations of a compound of the present disclosure and other active ingredients will generally also be within the aforementioned range, but in each case, an effective dose of each active ingredient should be used. In such combinations the compound of the present disclosure and other active agents may be administered separately or in conjunction. In addition, the administration of one element may be prior to, concurrent to, or subsequent to the administration of other agent(s).

A composition of the disclosure can be a liquid solution, suspension, emulsion, tablet, pill, capsule, sustained release formulation, or powder. The compositions can be formulated as a suppository, with traditional binders and carriers such as triglycerides. Oral formulations can include standard carriers such as pharmaceutical grades of mannitol, lactose, starch, magnesium stearate, sodium saccharine, cellulose, magnesium carbonate, etc. Various delivery systems are known and can be used to administer a composition of the disclosure, e.g. encapsulation in liposomes, microparticles, microcapsules, and the like.

A therapeutic composition of the disclosure may comprise a carrier, such as one or more of a polymer, carbohydrate, peptide or derivative thereof, which may be directly or indirectly covalently attached to the compound. A carrier may be substituted with substituents described herein including without limitation one or more alkyl, amino, nitro, halogen, thiol, thioalkyl, sulfate, sulfonyl, sulfinyl, sulfoxide, hydroxyl groups. In aspects of the disclosure the carrier is an amino acid including alanine, glycine, praline, methionine, serine, threonine, asparagine, alanyl-alanyl, prolyl-methionyl, or glycyl-glycyl. A carrier can also include a molecule that targets a compound of the disclosure to a particular tissue or organ.

Compounds of the disclosure can be prepared using reactions and methods generally known to the person of ordinary skill in the art, having regard to that knowledge and the disclosure of this application including the Examples. The reactions are performed in solvent appropriate to the reagents and materials used and suitable for the reactions being effected. It will be understood by those skilled in the art of organic synthesis that the functionality present on the compounds should be consistent with the proposed reaction steps. This will sometimes require modification of the order of the synthetic steps or selection of one particular process scheme over another in order to obtain a desired compound of the disclosure. It will also be recognized that another major consideration in the development of a synthetic route is the selection of the protecting group used for protection of the reactive functional groups present in the compounds described in this disclosure. An authoritative account describing the many alternatives to the skilled artisan is Greene and Wuts (Protective Groups In Organic Synthesis, Wiley and Sons, 1991).

A compound of the disclosure may be formulated into a pharmaceutical composition for administration to a subject by appropriate methods known in the art. Pharmaceutical compositions of the present disclosure or fractions thereof comprise suitable pharmaceutically acceptable carriers, excipients, and vehicles selected based on the intended form of administration, and consistent with conventional pharmaceutical practices. Suitable pharmaceutical carriers, excipients, and vehicles are described in the standard text, Remington: The Science and Practice of Pharmacy (21.sup.st Edition. 2005, University of the Sciences in Philadelphia (Editor), Mack Publishing Company), and in The United States Pharmacopeia: The National Formulary (USP 24 NF19) published in 1999. By way of example for oral administration in the form of a capsule or tablet, the active components can be combined with an oral, non-toxic pharmaceutically acceptable inert carrier such as lactose, starch, sucrose, methyl cellulose, magnesium stearate, glucose, calcium sulfate, dicalcium phosphate, mannitol, sorbital, and the like. For oral administration in a liquid form, the chug components may be combined with any oral, non-toxic, pharmaceutically, acceptable inert carrier such as ethanol, glycerol, water, and the like. Suitable binders (e.g., gelatin, starch, corn sweeteners, natural sugars including glucose; natural and synthetic gums, and waxes), lubricants (e.g. sodium oleate, sodium stearate, magnesium stearate, sodium benzoate, sodium acetate, and sodium chloride), disintegrating agents (e.g. starch, methyl cellulose, agar, bentonite, and xanthan gum), flavoring agents, and coloring agents may also be combined in the compositions or components thereof. Compositions as described herein can further comprise wetting or emulsifying agents, or pH buffering agents.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The terms “subject”, “individual”, or “patient” as used herein are used interchangeably and refer to an animal preferably a warm-blooded animal such as a mammal. Mammal includes without limitation any members of the Mammalia. A mammal, as a subject or patient in the present disclosure, can be from the family of Primates, Carnivora, Proboscidea, Perissodactyla, Artiodactyla, Rodentia, and Lagomorphaln an embodiment, animals can be treated; the animals can be vertebrates, including both birds and mammals. In aspects of the disclosure, the terms include domestic animals bred for food or as pets, including equines, bovines, sheep, poultry, fish, porcines, canines, felines, and zoo animals, goats, apes (e.g. gorilla or chimpanzee), and rodents such as rats and mice.

In a particular embodiment, the mammal is a human. In an aspect, the subject is a neonate. A neonate is an infant that is 28 days old or less from birth. In an aspect, the subject is an adult. An adult is a human that has reached puberty. In an aspect, the subject is a child. A child is a human between a neonate and an adult. Puberty can be described as a process (e.g., where the time frame varies among humans, in particular, males and females) where the body of a child matures into a body of an adult and is capable of sexual reproduction.

The term “pharmaceutically acceptable carrier” as used herein refers to a diluent, adjuvant, excipient, or vehicle with which a probe of the disclosure is administered, and which is approved by a regulatory agency of the Federal or a state government or listed in the U.S. Pharmacopeia or other generally recognized pharmacopeia for use in animals, and more particularly in humans. Such pharmaceutical carriers can be liquids, such as water and oils, including those of petroleum, animal, vegetable, or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. When administered to a patient, the probe and pharmaceutically acceptable carriers can be sterile. Water is a useful carrier when the probe is administered intravenously. Saline solutions and aqueous dextrose and glycerol solutions can also be employed as liquid carriers, particularly for injectable solutions. Suitable pharmaceutical carriers also include excipients such as glucose, lactose, sucrose, glycerol monostearate, sodium chloride, glycerol, propylene, glycol, water, ethanol and the like. The present compositions, if desired, can also contain minor amounts of wetting or emulsifying agents, or pH buffering agents. The present compositions advantageously may take the form of solutions, emulsion, sustained-release formulations, or any other form suitable for use. Pharmaceutically acceptable carriers may also include a dentifrice.

The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Additionally, the term “pharmaceutically acceptable” describes a material that is not biologically or otherwise undesirable, i.e., without causing an unacceptable level of undesirable biological effects or interacting in a deleterious manner.

As used herein, the terms “effective amount” and “amount effective” refer to an amount that is sufficient to achieve the desired result or to have an effect on an undesired disease, disorder, and/or condition or prevention of a disease, disorder, and/or condition or enhance and/or tune the immune system of the subject to the desirable responses to the disease, disorder, and/or condition. For example, a “therapeutically effective amount” refers to an amount that is sufficient to achieve the desired therapeutic result or to have an effect on undesired symptoms or prevention of a disease, disorder, and/or condition and/or tune the immune system of the subject to the desirable responses to the disease, disorder, and/or condition but is generally insufficient to cause adverse side effects. The specific therapeutically effective dose level for any particular patient will depend upon a variety of factors including; the specific composition employed; the age (e.g., neonate vs child vs adult), body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. It is known that the effective amount for treating a neonate can differ from the effective amount for treating a child and/or treating an adult. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

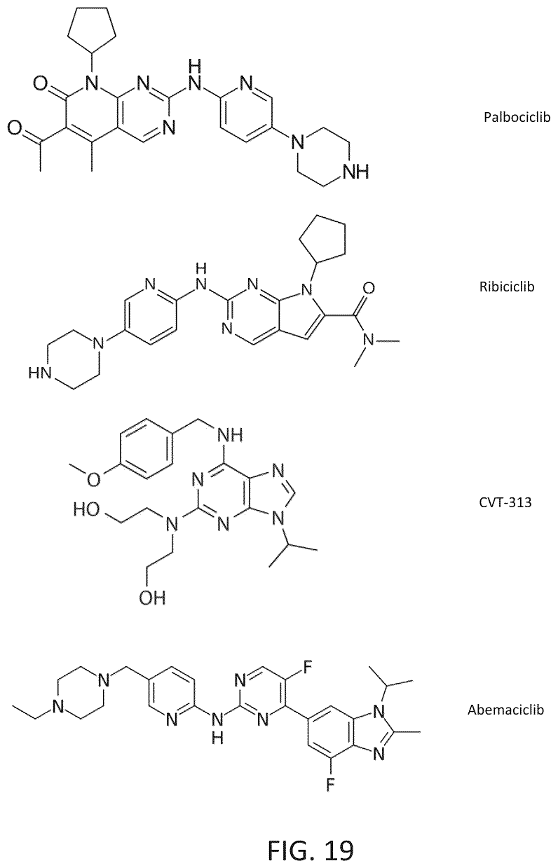

Palbociclib, also known as 6-acetyl-8-cyclopentyl-5-methyl-2-[5-(1-piperazinyl)pyridine-2-ylamino]pyrido[2,3-d]pyrimidin-7(8H)-one (See FIG. 19) and is marketed under the tradename Ibrance® Palbociclib, derivatives thereof, and pharmaceutically acceptable salts thereof are disclosed in International Publication No. WO 2003/062236 and U.S. Pat. Nos. 6,936,612, 7,208,489 and 7,456,168; International Publication No. WO 2005/005426 and U.S. Pat. Nos. 7,345,171 and 7,863,278; International Publication No. WO 2008/032157 and U.S. Pat. No. 7,781,583; and International Publication No. WO 2014/128588, where these describe methods of making palbociclib and derivatives thereof, salts thereof, and each are incorporated herein by reference in their entirety.

In an aspect, the therapeutically effective dose of palbociclib, salts thereof and derivatives thereof can vary from approximately 0.01 mg/kg to approximately 100 mg/kg of body weight per day. Typical adult doses will be approximately 0.1 mg to approximately 3000 mg per day. The quantity of active component in a unit dose preparation may be varied or adjusted from approximately 0.1 mg to approximately 500 mg, preferably about 0.6 mg to 100 mg according to the particular application and the potency of the active component. The composition can, if desired, also contain other compatible therapeutic agents. A subject in need of treatment with palbociclib, salts thereof and derivatives thereof can be administered a dosage of about 0.6 to about 500 mg per day, either singly or in multiple doses over a 24-hour period. Such treatment may be repeated at successive intervals for as long as necessary.

Ribociclib, also known as (7-cyclopentyl-N,N-dimethyl-2-[(5-piperazin-1-ylpyridin-2-yl)amino]pyrrolo[2,3-d]pyrimidine-6-carboxamide) (See FIG. 19), and derivatives thereof, and pharmaceutically acceptable salts thereof are disclosed WO 2010/020675, WO 2012/064805, WO 2016/166703, and WO 2007/140222, each are incorporated herein by reference in their entirety.

CVT-313 is also known as (2-[2-hydroxyethyl-[6-[(4-methoxyphenyl)methylamino]-9-propan-2-ylpurin-2-yl]amino]ethanol) (See FIG. 19) as described in U.S. Pat. Nos. 6,255,485, 6,617,331, 6,790,095, 6,803,371, 6,949,558, 7,109,330 each are incorporated herein by reference in their entirety.

Abemaciclib, also known as [5-(4-ethyl-piperazin-1-ylmethyl)-pyridin-2-yl]-[5-fluoro-4-(7-fluoro-3-isopropyl-2-methyl-3H-benzoimidazol-5-yl)-pyrimidin-2-yl]-amine (See FIG. 19), is sold under the name Verzenio® by Eli Lilly. Abemaciclib and derivatives thereof, and pharmaceutically acceptable salts thereof are disclosed U.S. Pat. No. 7,856,211 and WO2010/075074.

As used herein, the terms “treating” and “treatment” can refer generally to obtaining a desired pharmacological and/or physiological effect. The effect can be, but does not necessarily have to be, prophylactic in terms of preventing or partially preventing a disease, disorder, and/or condition, such as consequences thereof and/or tuning the immune system of the subject to the desirable responses to the disease, disorder, and/or condition. The effect can be therapeutic in terms of a partial or complete cure of a disease, disorder, and/or condition, or adverse effect attributed to the disease, disorder, and/or condition. The term “treatment” as used herein can include any treatment of diseases in a subject, particularly a human and can include any one or more of the following: (a) preventing the disease, disorder, and/or condition from occurring in a subject which may be predisposed (e.g., genetically predisposed) to the disease but has not yet been diagnosed as having it; (b) inhibiting the disease, disorder, and/or condition i.e., arresting its development; and (c) relieving the disease, disorder, and/or condition i.e., mitigating or ameliorating the disease, disorder, and/or condition and/or its symptoms or conditions, (d) and/or tune the immune system of the subject to the desirable responses for the disease, disorder, and/or condition. The term “treatment” as used herein can refer to both therapeutic treatment alone, prophylactic treatment alone, or both therapeutic and prophylactic treatment. In an aspect, treatment can be used to describe treatment of an adult having the disease, disorder, and/or condition. In an aspect, treatment can be used to describe prophylactic treatment of a neonate that is predisposed to the disease, disorder, and/or condition. In an aspect, treatment can be used to describe treatment of a child having the disease, disorder, and/or condition or to the treatment can be used to describe prophylactic treatment of a child that is predisposed to the disease, disorder, and/or condition. Those in need of treatment (subjects in need thereof) can include those already with the disease, disorder, and/or condition and/or those in which the disease, disorder, and/or condition is to be prevented. The disease, disorder, and/or condition can be a vascular malformation.

As used herein, “therapeutic” can refer to treating, healing, and/or ameliorating a disease, disorder, condition, or side effect, or to decreasing in the rate of advancement of a disease, disorder, condition, or side effect and/or tuning the immune system of the subject to the desirable responses. The disease, disorder, and/or condition can be a vascular malformation.

The term “pharmaceutically acceptable salts, esters, amides, and prodrugs” as used herein refers to those carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds described herein.

The term “salts” refers to the relatively non-toxic, inorganic and organic acid addition salts of compounds as described herein (e.g., cell cycle inhibitors such as palbociclib, ribociclib, CVT-313, and abemaciclib). These salts can be prepared in situ during the final isolation and purification of the compounds or by separately reacting the purified compound in its free base form with a suitable organic or inorganic acid and isolating the salt thus formed. In so far as the compounds described herein are basic compounds, they are all capable of forming a wide variety of different salts with various inorganic and organic acids. Although such salts must be pharmaceutically acceptable for administration to animals, it is often desirable in practice to initially isolate the base compound from the reaction mixture as a pharmaceutically unacceptable salt and then simply convert to the free base compound by treatment with an alkaline reagent and thereafter convert the free base to a pharmaceutically acceptable acid addition salt. The acid addition salts of the basic compounds are prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present disclosure.

Pharmaceutically acceptable base addition salts are formed with metals or amines, such as alkali and alkaline earth metal hydroxides, or of organic amines. Examples of metals used as cations are sodium, potassium, magnesium, calcium, and the like. Examples of suitable amines are N,N′-dibenzylethylenediamine, chloroprocaine, choline, diethanolamine, ethylenediamine, N-methylglucamine, and procaine.

The base addition salts of acidic compounds are prepared by contacting the free acid form with a sufficient amount of the desired base to produce the salt in the conventional manner. The free acid form may be regenerated by contacting the salt form with an acid and isolating the free acid in a conventional manner. The free acid forms differ from their respective salt forms somewhat in certain physical properties such as solubility in polar solvents, but otherwise the salts are equivalent to their respective free acid for purposes of the present invention.

Salts may be prepared from inorganic acids sulfate, pyrosulfate, bisulfate, sulfite, bisulfite, nitrate, phosphate, monohydrogenphosphate, dihydrogenphosphate, metaphosphate, pyrophosphate, chloride, bromide, iodide such as hydrochloric, nitric, phosphoric, sulfuric, hydrobromic, hydriodic, phosphorus, and the like. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, laurylsulphonate and isethionate salts, and the like. Salts may also be prepared from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. and the like. Representative salts include acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Pharmaceutically acceptable salts may include cations based on the alkali and alkaline earth metals, such as sodium, lithium, potassium, calcium, magnesium and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. Also contemplated are the salts of amino acids such as arginate, gluconate, galacturonate, and the like. (See, for example, Berge S. M. et al., “Pharmaceutical Salts,” J. Pharm. Sci., 1977; 66:1-19 which is incorporated herein by reference.)

Examples of pharmaceutically acceptable, non-toxic esters of the compounds as described herein include C₁-C₆ alkyl esters wherein the alkyl group is a straight or branched chain. Acceptable esters also include C₅-C₇ cycloalkyl esters as well as arylalkyl esters such as, but not limited to benzyl. C₁-C₄ alkyl esters are preferred. Esters of the compounds described herein may be prepared according to conventional methods “March's Advanced Organic Chemistry, 5^{th} Edition”. M. B. Smith & J. March, John Wiley & Sons, 2001.

Examples of pharmaceutically acceptable, non-toxic amides of the compounds as described herein include amides derived from ammonia, primary C₁-C₆ alkyl amines and secondary C₁-C₆ dialkyl amines wherein the alkyl groups are straight or branched chain. In the case of secondary amines the amine may also be in the form of a 5- or 6-membered heterocycle containing one nitrogen atom. Amides derived from ammonia, C₁-C₃ alkyl primary amines and C₁-C₂ dialkyl secondary amines are preferred. Amides of the compounds as described herein may be prepared according to conventional methods such as “March's Advanced Organic Chemistry. 5th Edition”. M. B. Smith & J. March, John Wiley & Sons, 2001.

The term “prodrug” refers to compounds that are rapidly transformed in vivo to yield the compound (e.g., palbociclib, ribociclib, CVT-313, and abemaciclib), for example, by hydrolysis in blood. A thorough discussion is provided in T. Higuchi and V. Stella, “Pro-drugs as Novel Delivery Systems.” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference.

### General Discussion

The present disclosure provides for methods of treating vascular malformation using cell cycle inhibitors, such as palbociclib, ribociclib, CVT-313, and abemaciclib. The methods can include treating adults that have developed a vascular malformation. In addition, the methods can include treating neonates that may develop a vascular malformation. Further, the methods can include treating children that have developed a vascular malformation or that may develop a vascular malformation. Additional details are provided herein and in the Examples.

The present disclosure, as described in detail in the Examples, illustrates that endothelial cell cycle state and cell cycle regulatory genes are dysregulated in preclinical models of hereditary hemorrhagic telangiectasia (HHT) induced by BMP9/10 blocking antibodies, as well as Alk1 genetic endothelial-specific inducible deletion in the Fucci2 cell cycle reporter mice. As described herein, cell cycle inhibitors, such as palbociclib, ribociclib, CVT-313, and abemaciclib, prevent vascular malformations induced by both BMP9/10 blocking antibodies and endothelial-specific loss of Alk1, referred to as Alk1EC^{iKO} Mechanistically, it has been found that CDK4/6 inhibition-induced endothelial cell cycle arrest enables the expression of genes that collectively prevent the dysregulation of arteriovenous identity, migration and metabolism, as well as genes regulating the VEGF-A and BMP9 signaling pathways, which are known to contribute to HHT pathogenesis. Thus, this information provides insights into molecular mechanisms leading to HHT by defining how endothelial cell cycle is dysregulated in AVMs due to BMP9/10 and Alk1 signaling deficiencies. It also shows that cell cycle inhibitors, such as palbociclib, ribociclib, CVT-313, and abemaciclib can be used for the treatment of vascular malformations in HHT patients. In addition, since dysregulation of endothelial cell cycle control is certainly not restricted to HHT, other diseases characterized by endothelial cell hyperproliferation and loss of identity such as vascular malformation (e.g., cerebral cavernous malformations and/or venous malformations) can be treated cell cycle inhibitors, such as palbociclib, ribociclib, CVT-313, and abemaciclib. Additional details are provided in the Examples.

There are multiple types of vascular malformations depending on the type of blood vessel affected: arterio-venous, venous, capillary, lymphatic and cerebral cavernous malformations (CCM). In most cases, these anomalies are linked to genetic mutations of signaling pathways controlling blood vessel formation. Vascular malformations are characterized by morphologically and functionally abnormal blood vessels that are prone to rupture causing hemorrhage and are potentially fatal. Depending on the vascular malformation type, the abnormalities can affect different organs and can be symptomatic from birth or have a later onset. Vascular malformations can include an aneurysm, an arteriovenous malformation, a cavernous malformation, a venous malformation, a lymphatic malformation, a capillary telangiectasia, a mixed vascular malformation, a spinal dural arteriovenous fistula, or a combination thereof. Vascular malformations can occur in organs such as the brain, heart, lung, kidney, liver, or pancreas.

Hereditary Hemorrhagic Telangiectasia (HHT) is an autosomal dominant disorder with high penetrance that is characterized by malformed blood vessels, particularly multifocal artenovenous malformations (AVMs).

The present disclosure provides for a method for treating a condition including administering to a subject in need thereof, a pharmaceutical composition including a therapeutically effective amount of any one of the cell cycle inhibitors or related pharmaceutical composition disclosed herein. The pharmaceutical composition can include: palbociclib or pharmaceutically acceptable salts thereof, ribociclib or pharmaceutically acceptable salts thereof: CVT-313 or pharmaceutically acceptable salts thereof, or abemaciclib, or pharmaceutically acceptable salts thereof.

In an aspect, the subject can be a human adult. The method includes administering a therapeutically effective amount of any one of the cell cycle inhibitors or related pharmaceutical composition disclosed herein to the adult. The adult has developed the vascular malformation, for example, arterio-venous malformations, cerebral cavernous malformations, venous malformations, or lymphatic malformations. The pharmaceutical composition can include: palbociclib or pharmaceutically acceptable salts thereof, ribociclib or pharmaceutically acceptable salts thereof; CVT-313 or pharmaceutically acceptable salts thereof; or abemaciclib, or pharmaceutically acceptable salts thereof.

In an aspect, the subject can be a human child. The method includes administering a therapeutically effective amount of any one of the cell cycle inhibitors or related pharmaceutical composition disclosed herein to the adult. In an aspect, the child has developed the vascular malformation, for example, arterio-venous malformations, cerebral cavernous malformations, venous malformations, or lymphatic malformations. In another aspect. The child is diagnosed as being predisposed (e.g., family history, genetic testing, etc.) to developing the vascular malformation, for example, arterio-venous malformations, cerebral cavernous malformations, venous malformations, or lymphatic malformations. The pharmaceutical composition can include: palbociclib or pharmaceutically acceptable salts thereof; ribociclib or pharmaceutically acceptable salts thereof, CVT-313 or pharmaceutically acceptable salts thereof, or abemaciclib, or pharmaceutically acceptable salts thereof.

In an aspect, the subject is a human neonate. The method includes administering a therapeutically effective amount of any one of the cell cycle inhibitors or related pharmaceutical composition disclosed herein to the neonate. The therapeutically effective amount of the cell cycle inhibitors or related pharmaceutical composition to treat a neonate and an adult are likely different based on age, development, stage of vascular malformation, and other known differences between a therapeutically effective amount for an adult and neonate. The neonate is diagnosed as being predisposed (e.g., family history, genetic testing, etc.) to developing the vascular malformation, for example, arterio-venous malformations, cerebral cavernous malformations, venous malformations, or lymphatic malformations. The method includes prophylactically treating the neonate that is predisposed to developing the vascular malformation. The pharmaceutical composition can include: palbociclib or pharmaceutically acceptable salts thereof, ribociclib or pharmaceutically acceptable salts thereof; CVT-313 or pharmaceutically acceptable salts thereof; or abemaciclib, or pharmaceutically acceptable salts thereof.

In another aspect, the present disclosure provides for a method to prevent or treat endothelial cell hyperproliferation comprising administering to a subject in need thereof a pharmaceutical composition. The pharmaceutical composition comprises a therapeutically effective amount of the pharmaceutical composition. The pharmaceutical composition includes: palbociclib or pharmaceutically acceptable salts thereof; a ribociclib or pharmaceutically acceptable salts thereof: a CVT-313 or pharmaceutically acceptable salts thereof; or abemaciclib, or pharmaceutically acceptable salts thereof. The subject can be an adult, a child, or a neonate and the therapeutically effective amount of the pharmaceutical composition used for each may differ. The adult has developed endothelial cell hyperproliferation. The neonate may be predisposed to develop the endothelial cell hyperproliferation and the treatment can be prophylactic treatment. The child has developed endothelial cell hyperproliferation or may be predisposed to develop the endothelial cell hyperproliferation and the treatment can be prophylactic treatment.

In aspect, the present disclosure provides for a method to prevent or treat hereditary hemorrhagic telangiectasia (HHT) comprising administering to a subject in need thereof a pharmaceutical composition. The pharmaceutical composition comprises a therapeutically effective amount of the pharmaceutical composition. The pharmaceutical composition includes: palbociclib or pharmaceutically acceptable salts thereof; a ribociclib or pharmaceutically acceptable salts thereof; a CVT-313 or pharmaceutically acceptable salts thereof; or abemaciclib, or pharmaceutically acceptable salts thereof. The subject can be an adult, child, or a neonate and the therapeutically effective amount of the pharmaceutical composition used for each may differ. The adult has developed HHT. The neonate may be predisposed to develop HHT and the treatment can be prophylactic treatment. The child has developed HHT or may be predisposed to develop HHT and the treatment can be prophylactic treatment.

The present disclosure includes pharmaceutical compositions comprising a therapeutically effective amount of one or more disclosed compounds (e.g., cell cycle inhibitors), or pharmaceutically acceptable salt thereof, and a pharmaceutically acceptable carrier.

In a particular aspect, the pharmaceutical composition can include a therapeutically effective amount of a compound to prophylactically treat a vascular malformation in a neonate. The pharmaceutical composition includes: palbociclib or pharmaceutically acceptable salts thereof; ribociclib or pharmaceutically acceptable salts thereof; CVT-313 or pharmaceutically acceptable salts thereof; or abemaciclib, or pharmaceutically acceptable salts thereof.

In another aspect, the pharmaceutical composition includes a therapeutically effective amount of a compound to treat vascular malformation in an adult. The pharmaceutical composition include: palbociclib or pharmaceutically acceptable salts thereof; ribociclib or pharmaceutically acceptable salts thereof; CVT-313 or pharmaceutically acceptable salts thereof, or abemaciclib, or pharmaceutically acceptable salts thereof.

In a particular aspect, the pharmaceutical composition can include a therapeutically effective amount of a compound to treat or prophylactically treat a vascular malformation in a child. The pharmaceutical composition includes: palbociclib or pharmaceutically acceptable salts thereof; ribociclib or pharmaceutically acceptable salts thereof: CVT-313 or pharmaceutically acceptable salts thereof; or abemaciclib, or pharmaceutically acceptable salts thereof.

### Pharmaceutical Formulations and Routes of Administration

Embodiments of the present disclosure include the agent (e.g., cell cycle inhibitors such as palbociclib, ribociclib, CVT-313, and abemaciclib) as identified herein and can be formulated with one or more pharmaceutically acceptable excipients, diluents, carriers and/or adjuvants. In addition, embodiments of the present disclosure include the agent formulated with one or more pharmaceutically acceptable auxiliary substances. In particular the agent can be formulated with one or more pharmaceutically acceptable excipients, diluents, carriers, and/or adjuvants to provide an embodiment of a composition of the present disclosure.

A wide variety of pharmaceutically acceptable excipients are known in the art. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In an embodiment of the present disclosure, the agent can be administered to the subject using any means capable of resulting in the desired effect. Thus, the agent can be incorporated into a variety of formulations for therapeutic administration. For example, the agent can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, the agent may be administered in the form of its pharmaceutically acceptable salts, or a subject active composition may be used alone or in appropriate association, as well as in combination, with other pharmaceutically active compounds. The following methods and excipients are merely exemplary and are in no way limiting.

For oral preparations, the agent can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Embodiments of the agent can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Embodiments of the agent can be utilized in aerosol formulation to be administered via inhalation. Embodiments of the agent can be formulated into pressurized acceptable propellants such as dichlorodifluoromethane, propane, nitrogen and the like.

Furthermore, embodiments of the agent can be made into suppositories by mixing with a variety of bases such as emulsifying bases or water-soluble bases. Embodiments of the agent can be administered rectally via a suppository. The suppository can include vehicles such as cocoa butter, carbowaxes and polyethylene glycols, which melt at body temperature, yet are solidified at room temperature.

Unit dosage forms for oral or rectal administration, such as syrups, elixirs, and suspensions, may be provided wherein each dosage unit, for example, teaspoonful, tablespoonful, tablet or suppository, contains a predetermined amount of the composition containing one or more compositions. Similarly, unit dosage forms for injection or intravenous administration may comprise the agent in a composition as a solution in sterile water, normal saline or another pharmaceutically acceptable carrier.

Embodiments of the agent can be formulated in an injectable composition in accordance with the disclosure. Typically, injectable compositions are prepared as liquid solutions or suspensions; solid forms suitable for solution in, or suspension in, liquid vehicles prior to injection may also be prepared. The preparation may also be emulsified or the active ingredient (triamino-pyridine derivative and/or the labeled triamino-pyridine derivative) encapsulated in liposome vehicles in accordance with the present disclosure.

In an embodiment, the agent can be formulated for delivery by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present disclosure. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, delivery of the agent can be accomplished using any of a variety of refillable, pump systems. Pumps provide consistent, controlled release over time. In some embodiments, the agent can be in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to, a subdermal, subcutaneous, intramuscular, or other suitable site within a subject's body. Subcutaneous implantation sites are used in some embodiments because of convenience in implantation and removal of the drug delivery device.

Drug release devices suitable for use in the disclosure may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present disclosure. Examples of such devices include those described in, for example, U.S. Pat. Nos. 4,692,147; 4,360,019; 4,487,603; 4,360,019; 4,725,852, and the like. In general, a subject treatment method can be accomplished using any of a variety of refillable, non-exchangeable pump systems. Pumps and other convective systems are generally preferred due to their generally more consistent, controlled release over time. Osmotic pumps are used in some embodiments due to their combined advantages of more consistent controlled release and relatively small size (see, e.g., PCT published application no. WO 97/27840 and U.S. Pat. Nos. 5,985,305 and 5,728,396). Exemplary osmotically-driven devices suitable for use in the disclosure include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like.

In some embodiments, the drug delivery device is an implantable device. The drug delivery device can be implanted at any suitable implantation site using methods and devices well known in the art. As noted herein, an implantation site is a site within the body of a subject at which a drug delivery device is introduced and positioned. Implantation sites include, but are not necessarily limited to a subdermal, subcutaneous, intramuscular, or other suitable site within a subject's body.

In some embodiments, the agent can be delivered using an implantable drug delivery system, e.g., a system that is programmable to provide for administration of the agent. Exemplary programmable, implantable systems include implantable infusion pumps. Exemplary implantable infusion pumps, or devices useful in connection with such pumps, are described in, for example, U.S. Pat. Nos. 4,350,155; 5,443,450; 5,814,019; 5,976,109; 6,017,328; 6,171,276; 6,241,704; 6,464,687; 6,475,180; and 6,512,954. A further exemplary device that can be adapted for the present disclosure is the Synchromed infusion pump (Medtronic).

Suitable excipient vehicles for the agent are, for example, water, saline, dextrose, glycerol, ethanol, or the like, and combinations thereof. In addition, if desired, the vehicle may contain minor amounts of auxiliary substances such as wetting or emulsifying agents or pH buffering agents. Methods of preparing such dosage forms are known, or will be apparent upon consideration of this disclosure, to those skilled in the art. See, e.g., Remington's Pharmaceutical Sciences, Mack Publishing Company, Easton, Pennsylvania, 17th edition, 1985. The composition or formulation to be administered will, in any event, contain a quantity of the agent adequate to achieve the desired state in the subject being treated.

Compositions of the present disclosure can include those that comprise a sustained-release or controlled release matrix. In addition, embodiments of the present disclosure can be used in conjunction with other treatments that use sustained-release formulations. As used herein, a sustained-release matrix is a matrix made of materials, usually polymers, which are degradable by enzymatic or acid-based hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxcylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. Illustrative biodegradable matrices include a polylactide matrix, a polyglycolide matrix, and a polylactide co-glycolide (co-polymers of lactic acid and glycolic acid) matrix.

In another embodiment, the pharmaceutical composition of the present disclosure can be delivered in a controlled release system. For example, the agent may be administered using intravenous infusion, an implantable osmotic pump, a transdermal patch, liposomes, or other modes of administration. In one embodiment, a pump may be used (Sefton (1987). CRC Crit. Ref. Biomed. Eng. 14:201; Buchwald et al. (1980). Surgery 88:507; Saudek et al. (1989). N. Engl. J. Med. 321:574). In another embodiment, polymeric materials are used. In yet another embodiment a controlled release system is placed in proximity of the therapeutic target thus requiring only a fraction of the systemic dose. In yet another embodiment, a controlled release system is placed in proximity of the therapeutic target, thus requiring only a fraction of the systemic. Other controlled release systems are discussed in the review by Langer (1990). Science 249:1527-1533.

In another embodiment, the compositions of the present disclosure include those formed by impregnation of the agent described herein into absorptive materials, such as sutures, bandages, and gauze, or coated onto the surface of solid phase materials, such as surgical staples, zippers and catheters to deliver the compositions. Other delivery systems of this type will be readily apparent to those skilled in the art in view of the instant disclosure.

### Dosages

Embodiments of the agent (e.g., cell cycle inhibitors) can be administered to a subject in one or more doses. Those of skill will readily appreciate that dose levels can vary as a function of the specific the agent administered, the severity of the symptoms and the susceptibility of the subject to side effects as well as the age of the subject. Preferred dosages for a given compound are readily determinable by those of skill in the art by a variety of means.

In an embodiment, multiple doses of the agent are administered. The frequency of administration of the agent can vary depending on any of a variety of factors, e.g., severity of the symptoms, the age of the subject, and the like. For example, in an embodiment, the agent can be administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

The duration of administration of the agent, e.g., the period of time over the agent is administered, can vary, depending on any of a variety of factors, e.g., subject response, subject age, etc. For example, the agent in combination or separately, can be administered over a period of time of about one day to one week, about two weeks to four weeks, about one month to two months, about two months to four months, about four months to six months, about six months to eight months, about eight months to 1 year, about 1 year to 2 years, or about 2 years to 4 years, or more.

### Routes of Administration

Embodiments of the present disclosure provide methods and compositions for the administration of the agent (e.g., cell cycle inhibitors) to a subject (e.g., a human) using any available method and route suitable for drug delivery, including in vivo and ex vivo methods, as well as systemic and localized routes of administration.

Routes of administration include intranasal, intramuscular, intratracheal, subcutaneous, intradermal, topical application, intravenous, rectal, nasal, oral, and other enteral and parenteral routes of administration. Routes of administration may be combined, if desired, or adjusted depending upon the agent and/or the desired effect. An agent can be administered in a single dose or in multiple doses.

Embodiments of the agent can be administered to a subject using available conventional methods and routes suitable for delivery of conventional drugs, including systemic or localized routes. In general, routes of administration contemplated by the disclosure include, but are not limited to, enteral, parenteral, or inhalational routes.

Parenteral routes of administration other than inhalation administration include, but are not limited to, topical, transdermal, subcutaneous, intramuscular, intraorbital, intracapsular, intraspinal, intrasternal, and intravenous routes, i.e., any route of administration other than through the alimentary canal. Parenteral administration can be conducted to effect systemic or local delivery of the agent. Where systemic delivery is desired, administration typically involves invasive or systemically absorbed topical or mucosal administration of pharmaceutical preparations.

In an embodiment, the agent can also be delivered to the subject by enteral administration. Enteral routes of administration include, but are not limited to, oral and rectal (e.g., using a suppository) delivery.

Methods of administration of the agent through the skin or mucosa include, but are not limited to, topical application of a suitable pharmaceutical preparation, transdermal transmission, injection and epidermal administration. For transdermal transmission, absorption promoters or iontophoresis are suitable methods. Iontophoretic transmission may be accomplished using commercially available “patches” that deliver their product continuously via electric pulses through unbroken skin for periods of several days or more.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

### Example 1

Hereditary Hemorrhagic Telangiectasia (HHT), is a rare autosomal-dominant vascular disease characterized by telangiectases and larger vascular malformations, that affects 1 in 6,000 children and adults worldwide¹. The known HHT-causing mutations affect genes encoding different components of the Bone Morphogenic Protein (BMP) 9 and BMP10 signaling pathway, namely: ENG, encoding the membrane glycoprotein ENDOGLIN in HHT type 1 and ACVRL1, encoding the membrane receptor ALK1 in HHT type 2^{2,3}. These mutations are detected in approximately 90% of cases submitted for molecular diagnosis.

HHT causes abnormal connections to develop between arteries and veins, known as arteriovenous malformations (AVMs). The most common organs affected by vascular malformations in HHT patients are the nose, skin, lungs, brain, and liver. In the nose and skin, the vascular malformations are referred to as telangiectases which are abnormal direct communications between dilated arterioles and postcapillary venules disrupting the capillary bed². These lesions are prone to rupture and cause uncontrolled nose bleeding that can ultimately result in chronic anemia. In the lungs, brain, intestinal tract and liver, larger AVMs are formed and can lead to life-threatening pulmonary hemorrhage, stroke, intestinal bleeding or liver failure^{1,2}. Pulmonary and brain AVMs are more common in patients with HHT type 1, while hepatic vascular malformations are more frequent in HHT type 2^{1,3}. Symptoms often begin during childhood and progress in severity. However, most patients typically do not receive a definitive HHT diagnosis until >40 years of age since they do not display any warning signs before AVMs rupture and/or do not receive genetic counseling or testing for HHT.

Vascular endothelial cells (ECs) lining blood vessels are the primary cells affected in HHT³. In ECs, decreased activity of BMP9/10 signaling leads to over-activation of the proangiogenic factors VEGF-A and angiopoietin-2 (ANGPT2)⁴, triggering endothelial cell hyperproliferation, as well as alterations in their permeability and migration⁵, ultimately leading to vascular malformations. Preclinical models employing postnatal endothelial-specific homozygous inducible-deletion of any of these genes, or pharmacological inhibition of the BMP9/10 signaling pathway via blocking antibodies, induces HHT-like vascular malformations, including excessive angiogenesis, enlarged veins and AV shunts in the murine retinal vascularization model⁵. While aberrant endothelial proliferation has been associated with loss of arteriovenous identity and AVMs³, the mechanistic link between endothelial cell cycle control and the development of AVMs has not been investigated, and this is the focus of our study.

Cell cycle progression is highly regulated by checkpoints that maintain cells in distinct phases of the cell cycle. Cells divide during mitosis, or M phase, and proceed to the first gap or G1 phase, where there is a checkpoint that can stall cells in early G1^{6,7}. A later G1-S checkpoint maintains cells in late G1. Our group recently showed that during development and in adulthood, venous ECs are predominantly in early G1, while arterial ECs are predominantly in late G1, providing distinct “windows of opportunity” for responses to extrinsic signals that promote changes in gene expression that enables EC specification⁸. We also demonstrated that the early G1 state is essential for BMP4-induced venous genes; whereas late G1 state is essential for TGF-β1-induced arterial gene expression⁸. Since endothelial proliferation is often dysregulated in AVMs, in conjunction with loss of endothelial identity, this study investigates whether dysregulated endothelial cell cycle control contributes to AVM development and if cell cycle modulators can be used to prevent or regress them.

Herein, we show that endothelial cell cycle state and cell cycle regulatory genes are dysregulated in preclinical models of HHT induced by BMP9/10 blocking antibodies, as well as Alk1 genetic endothelial-specific inducible deletion in the Fucci2 cell cycle reporter mice⁹. Furthermore, we demonstrated that Palbociclib¹⁰, an FDA-approved inhibitor of CDK4 and CDK6 currently used in clinical trials for breast cancer treatment¹¹, prevents vascular malformations induced by both BMP9/10 blocking antibodies and endothelial-specific loss of Alk1, referred to as Alk1EC^{iKO}. Mechanistically, we found that CDK4/6 inhibition-induced endothelial cell cycle arrest enables the expression of genes that collectively prevent the dysregulation of arteriovenous identity, migration and metabolism, as well as genes regulating the VEGF-A and BMP9 signaling pathways, which are known to contribute to HHT pathogenesis.

### Methods:

The data and methods supporting this study's findings are available from the corresponding authors on request. The single cell RNA seq data have been deposited at Gene Expression Omnibus database (GEO). The accession number for the dataset is available on request from the corresponding authors. Expanded methods are available in the Supplemental Material.

### Animals:

Male and female mice were used to minimize gender-related biased results. All animal protocols and procedures were reviewed and approved by the University of Virginia Animal Care and Use Committee (protocol #4277) and complied with all ethical regulations. The expanded methods in the Supplement Material contain a list of all mouse strains and protocols.

### Data Analysis and Statistics:

All continuous variables were represented as mean±SEM. The Mann-Whitney non-parametric test for unpaired samples was used to analyze continuous variables between groups. One-way ANOVA with Holm-Sidak's multiple comparisons test was used to compare the means of continuous variables among 3 groups. The Two-way ANOVA parametric test was used to compare two or more groups to determine whether there is an interactive effect between two independent variables (control samples vs. treated samples) on a continuous dependent variable (polarity or time) (IBM SPSS Statistics). All graphs and analyses were generated using Prism 8.0 software (GraphPad).

### Results

### Endothelial Cell Cycle State and RNA Expression are Dysregulated in HHT.

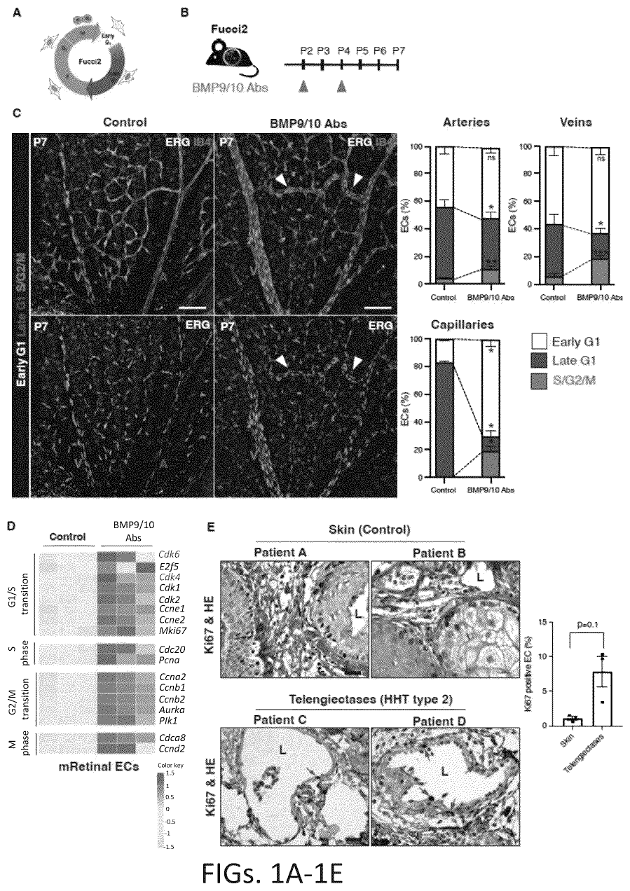

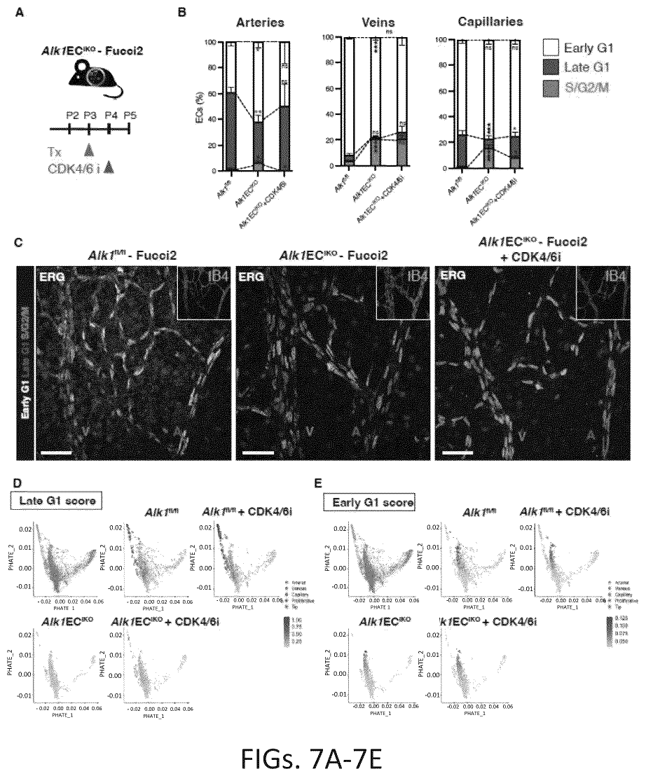

To analyze endothelial cell cycle state in AVMs, we first used a preclinical mouse model of HHT that employs BMP9/10 blocking antibodies to create arteriovenous shunts^{12,13}. BMP9/10 blocking antibodies were injected intraperitoneally (i.p.) at postnatal day (P)2 and P4 in mice expressing the Fucci2 cell cycle reporter, which enables dynamic visualization and quantification among ECs residing in the different cell cycle states (FIGS. 1A and B). For example, ECs in S/G2/M phases harbor green fluorescing nuclei, while EC nuclei in early G1 state are reporter-negative, and EC nuclei in late G1 state are fluorescing red. Consistent with our previous findings⁸, we found that in P7 control retinas, ECs forming veins and arteries predominantly reside in early G1 and late G1 state of the cell cycle, respectively (FIG. 1C). Conversely, in anti-BMP9/10-treated pups, we found a lower proportion of retinal arterial ECs in late G1, and significantly more ECs actively cycling in S/G2/M in both arteries and veins, compared to controls. In addition, among ECs forming the capillary plexi, we detected a significantly higher proportion of actively cycling cells in S/G2/M and in early G1 and, concomitantly, significantly less in late G1, compared to control (FIG. 1C). In addition, we performed EdU incorporation studies to identify actively proliferating ECs that are undergoing DNA synthesis. In anti-BMP9/10-treated pups, we found a significant higher number of EdU-positive retinal ECs in arterial, venous, and capillary vessels, and in distal vascular plexi above arterial and venous vessels, compared to controls. Altogether, these results show a dysregulation of cell cycle state and hyperproliferation of all endothelial subtypes in HHT (FIG. 9A-9E).

To further characterize the dysregulation of endothelial cell cycle state, we performed bulk RNA sequencing of murine primary retinal ECs isolated from P7 control and anti-BMP9/10-treated pups. Gene Ontology (GO) analysis revealed that mRNA expression of gene families regulating cell cycle progression and proliferation was highly enriched in the anti-BMP9/10-treated group (FIG. 9E). More specifically, analysis of differentially expressed genes in retinal ECs isolated from anti-BMP9/10-treated tissues showed upregulation of several genes that promote G1-to-S cell cycle transition such as CDK4, CDK6 and Mki67, as well as genes that regulate S/G2/M phases (FIG. 1D).

Finally, endothelial cell cycle status was evaluated in human dermal telangiectases from three patients with HHT type 2, and control normal skin biopsies in resection borders from three patients with melanoma. Using immunohistochemistry staining for Ki67, a protein expressed during S/G2/M phases, we found a higher number of Ki67-positive ECs in dermal telangiectases from HHT type 2 patients compared to normal skin samples (FIG. 9E), which is consistent with previous studies¹⁴. Collectively, these results demonstrate that endothelial cell cycle is dysregulated in both preclinical and clinical HHT conditions leading to EC hyperproliferation. Our data suggest that drugs that induce endothelial cell cycle arrest could be clinically relevant for the treatment of HHT patients to prevent or regress vascular malformations.

### Palbociclib Prevents AVMs Induced by BMP9/10 Blocking Antibodies by Promoting Endothelial Cell Cycle Late G1 State.

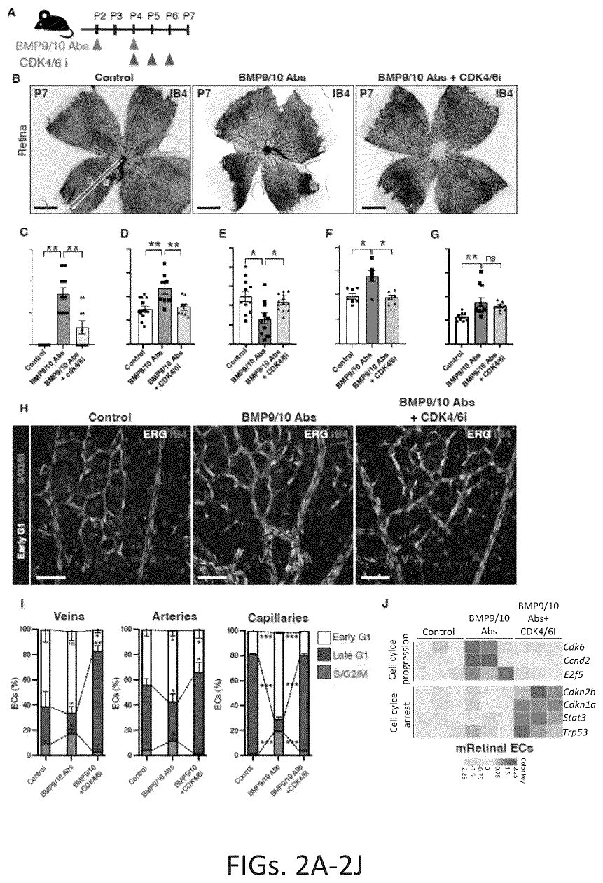

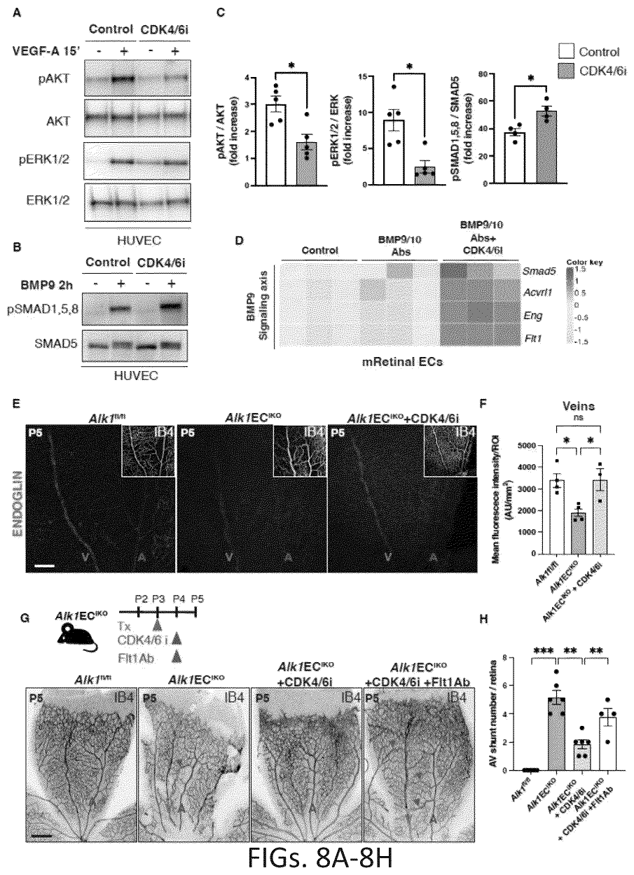

We found that CDK4 and CDK6 are upregulated in ECs isolated from anti-BMP9/10-treated mice (FIG. 1D). Thus, to test whether the use of pharmacological cell cycle modulators could prevent the development of AVMs in a murine model of HHT, we used Palbociclib, a CDK4 and CDK6 inhibitor (CDK4/6i) that blocks the transition from G1 to S state⁸. Pups were injected at P2 and P4 with BMP9/10 antibodies, followed by Palbociclib treatment starting at P4, then at P5 and P6 (FIG. 2A). At P7, we found, as expected, that mice receiving BMP9/10 antibodies, exhibited AV shunts and hyperdense retinal vascular plexi, compared to control tissues (FIG. 2B-2D). However, pups receiving both BMP9/10 blocking antibodies and CDK4/6i develop fewer AV shunts and have less hyperdense retinal vascular plexi, and CDK4/6i treatment normalized retinal vascular progression and venous vessel diameters but not arterial vessel diameters (FIG. 2B-G). Importantly, the CDK4/6i treatment by itself did not impair retinal vascular development in Palbociclib treated P7 control pups (FIGS. 10A and 10B).

We then analyzed the effects of BMP9/10 antibodies and CDK4/6i treatment on lungs, brains, and intestinal tracts, which are commonly affected by vascular malformations in HHT patients. To visualize vascular anomalies in these tissues, we performed intracardiac injections of blue latex silicon dye in P7 control, and anti-BMP9/10-treated pups, with or without CDK4/6i treatment following the dosing protocol as shown in FIG. 2A. In the lungs of anti-BMP9/10-treated animals, blue latex dye injections revealed vasodilated intrapulmonary arteries of 1^{st}, 2^{nd} and 3^{rd} order and perfusion of a dense network of disorganized capillaries, compared to controls (FIG. 10C). Anti-BMP9/10-treated animals that received CDK4/6i treatment exhibited significantly less vascular anomalies and intrapulmonary microvasculature vasodilation (FIG. 10D). Similarly, in the intestinal tracts, we found significant vasodilation of the small capillaries perfusing the duodenum in anti-BMP9/10-treated mice that was not prevented by CDK4/6i treatment during the dosing timeframe used herein (FIGS. 10E and 10F). Conversely, in the brains of anti-BMP9/10-treated animals, we did not detect defects in the cerebral vasculature, notably in the middle cerebral artery (MCA) and basilar artery (BA) (FIGS. 10G and 10H). Taken together, our data show that treatment with BMP9/10 antibodies induces significant vascular anomalies, particularly in the lungs and intestinal tracts. Interestingly, CDK4/6i treatment shows a preventive effect only in the lungs.

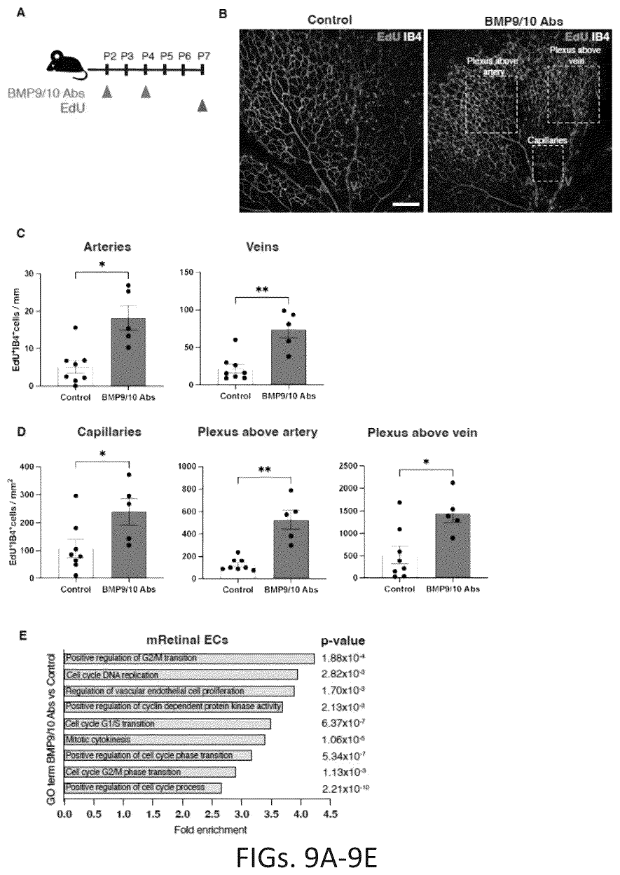

Next, we evaluated the effects of CDK4/6i in mice expressing the Fucci2 cell cycle reporter following the same dosing protocol outlined in FIG. 2A. BMP9/10 blocking antibody treatment caused a significantly higher proportion of actively cycling ECs in S/G2/M phases in arteries, veins and capillaries (FIG. 2H-I), as well as in the distal plexi (FIG. 11A), compared to controls, consistent with data shown in FIG. 1C. In pups treated with CDK4/6i, following BMP9/10 blocking antibody treatment, vascular malformations were absent, and ECs in all vessel types were predominantly in late G1 state (FIG. 2H-I). Since Palbociclib is a known cell cycle modulator, we assessed its effects on endothelial cell cycle state in the retinal vasculature. First, bulk RNA sequencing analysis revealed that mRNA levels of key genes promoting cell cycle progression, including Cond2 (CyclinD2), Cdk6 and E2f5, were highly enriched in retinal ECs isolated from anti-BMP9/10-treated pups. In contrast, mRNA levels of genes that promote cell cycle arrest were highly enriched in ECs from pups receiving BMP9/10 antibodies, followed by CDK4/6i, such as Cdkn2b (P15), Trp53 (P53) and Cdkn1a (P21) (FIG. 2J). Furthermore, as previously shown¹⁵, CDK4/6i treatment of human vein endothelial cells (HUVECs) induced hypo-phosphorylation of the Retinoblastoma protein (Rb) (FIG. 11B), which is known to induce cell cycle arrest by inhibition of E2F transcription factor activity¹⁵ (FIG. 11C). These results suggest that Palbociclib prevents ECs hyperproliferation and AV shunt formation by enabling the expression of genes that collectively promote endothelial late G1 state (as schematized in FIG. 11C).

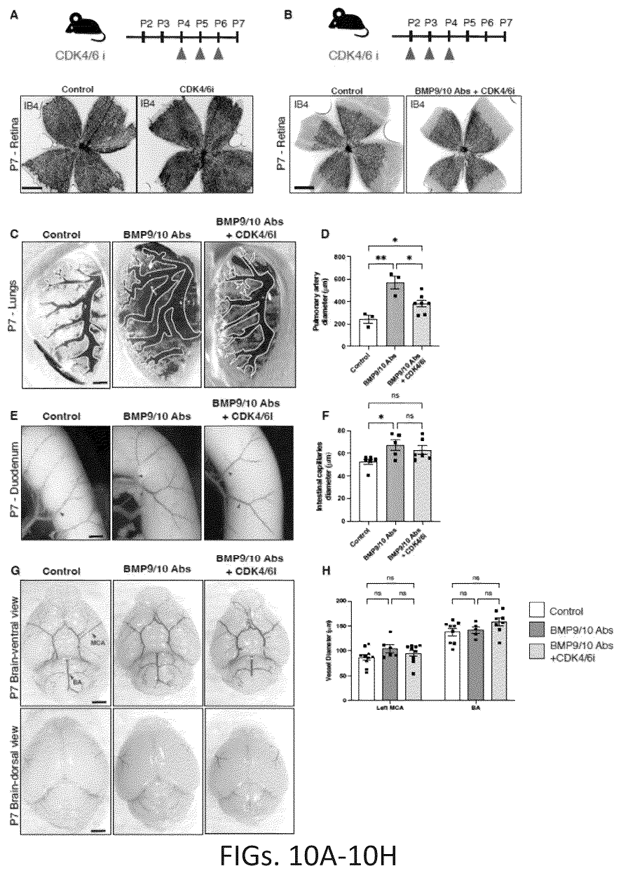

Finally, we assessed the effects of Palbociclib treatment on arteriovenous identity in anti-BMP9/10-treated animals following the dosing protocol shown in FIG. 12A. We found that Jagged1 (JAG1) protein expression is enriched in arterial ECs in control animals and is decreased in anti-BMP9/10-treated retinal vasculature (FIGS. 12B and 12C). JAG1 protein expression was increased in arterial ECs in animals that received CDK4/6i, following BMP9/10 antibody treatment, compared to animals that received only BMP9/10 antibodies (FIGS. 12B and 12C). These results are consistent with our previous studies showing that endothelial late G1 state is required to enable arterial specification¹⁶ and, herein, prevent arterial specification defects. Additionally, retinal arteries in anti-BMP9/10-treated animals exhibited reduced smooth muscle cell (SMC) coverage, as assessed by alpha smooth muscle actin (aSMA)-expressing cells, compared to control tissues. Interestingly, CDK4/6i treatment prevented the loss of SMC coverage on arteries (FIGS. 12D and 12E). Finally, ENDOMUCIN, which is enriched in veins and capillaries in control retinas, was ectopically expressed in arteries and AV shunts in the retinal vasculature of anti-BMP9/10-treated animals, compared to controls (FIGS. 12F and 12G). However, CDK4/6 inhibition did not prevent the arterial expression of ENDOMUCIN (FIGS. 12F and 12G) during the short window of time between the end of treatment and the collection of retinas for analysis. Overall, these results demonstrate that endothelial late G1 cell cycle state, induced by Palbociclib treatment, prevents AVM formation and loss of arteriovenous identity in HHT.

### Induced-Late G1 Endothelial Cell Cycle State Promotes Polarized Migration.

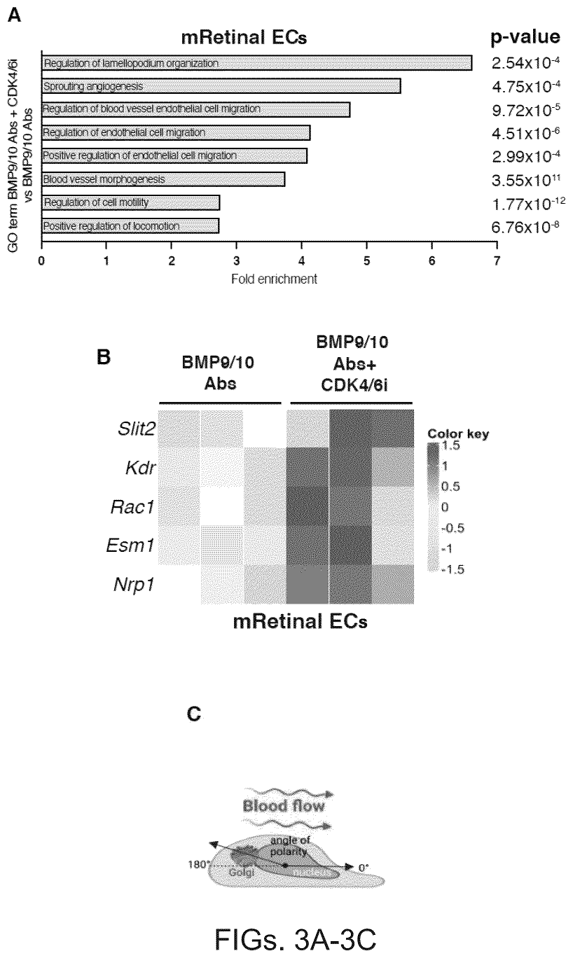

Other studies reported that defective endothelial cell migration is involved in the genesis of AV shunts in HHT mouse models^{17,18}. Herein, we performed Gene Ontology analysis of bulk RNA sequencing data from retinal ECs isolated from P7 pups treated with anti-BMP9/10, with or without CDK4/6i, as in FIG. 2A. We found enriched expression of several gene families that regulate cell migration in retinal ECs isolated from anti-BMP9/10- and CDK4/6i-treated animals, compared to mice that received only BMP9/10 antibody treatment (FIG. 3A). Further analysis of differentially expressed genes revealed enrichment of genes, such as Slit2, Kdr, Rac1, Nrp1 and Esm1, which are known to control endothelial cell migration and angiogenesis^{19,20,21} (FIG. 3B), in retinal ECs isolated from animals treated with anti-BMP 9/10 and CDK4/6i.

To assess flow-mediated EC polarized migration in response to anti-BMP9/10, with or without CDK4/6i, we co-immunostained P7 retinas with anti-GM130, a peripheral membrane protein located in the cis-Golgi, and IB4 and anti-ERG1,2,3 to label ECs. To analyze the orientation of the Golgi toward the direction of the flow in retinal vessels, we measured the angle formed by the vector nucleus/Golgi in each EC and the predicted blood flow vector in the vessel (as depicted in FIG. 3C). We also quantified the EC polarization using a polarity index²² ranging from 1 (highly polarized) to 0 (random distribution). As previously described^{17,22}, in control retinas, both venous and arterial ECs are oriented in the opposite direction of blood flow, with the Golgi positioned in front of the nuclei (FIGS. 3D, E and F). In retinas from mice treated with BMP9/10 antibodies, venous ECs exhibited a non-polarized pattern toward blood flow (FIGS. 3D, E and F). However, in mice treated with anti-BMP9/10 and CDK4/6i, the polarity of retinal ECs in veins and arteries was oriented similarly to controls (FIGS. 3D, E and F). Altogether, these results demonstrate that late G1 state induced by CDK4/6 inhibition promotes the expression of genes regulating EC migration and prevents EC migration defects caused by BMP9/10 deficiency.

### Induced-Late G1 Endothelial Cell Cycle State Prevents Pathological Metabolic Rewiring.

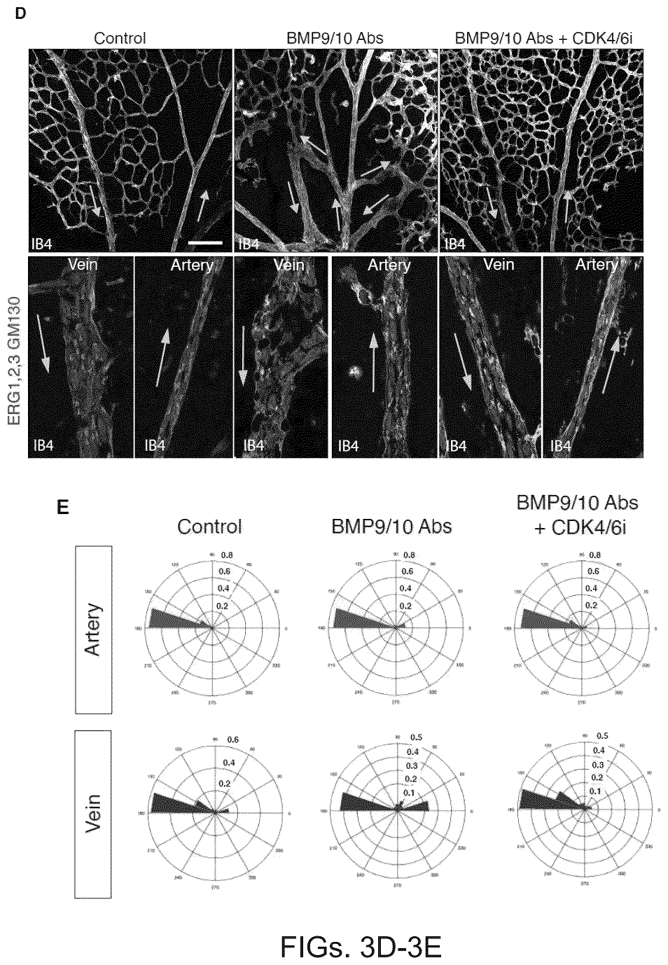

Previous studies revealed that specific metabolic pathways control EC function and behavior²³. For example, quiescent or proliferating ECs employ different metabolic pathways for energy production. A metabolic rewiring towards glycolysis and mitochondrial pathways occurs in ECs when they are activated during both physiological and pathological angiogenesis. Additionally, it has been shown that activation of specific metabolic pathways is associated with cell cycle progression²⁴. However, such metabolic changes in ECs during the formation of AVMs have not been investigated. RNA sequencing analysis of P7 retinal ECs isolated from control and anti-BMP9/10-treated mice revealed enriched mRNA expression of genes that regulate glycolysis, mitochondrial energy production through the TCA (Tricarboxylic acid or Kreb's) cycle and fatty acid signaling in response to anti-BMP9/10 treatment (FIG. 4A), which is consistent with increased ECs energy production/consumption associated with AVM development. Interestingly, we found that late G1 cell cycle state induced by Palbociclib treatment led to decreased mRNA expression of genes controlling glycolysis, TCA cycle and fatty acid pathways in retinal ECs (FIG. 4B).

Finally, we analyzed the effect of CDK4/6 inhibition on the metabolic activity of HUVECs using Agilent Seahorse Assay. The mitochondrial stress test was performed measuring basal oxygen consumption rate (OCR) (basal respiration), and after injection of 1 mM oligomycin, 2 mM BAM 15 (respiratory capacity) and 10 mM antimycin A and 1 mM rotenone (non-mitochondrial oxygen consumption, used for normalization) (FIG. 4C). We found that cells pretreated with Palbociclib (3 mg/ml) prior to analysis exhibited significantly lower mitochondrial basal respiration and respiratory capacity, compared to control cells (FIG. 4C-E). These results correlate with the inhibition of genes regulating mitochondrial TCA cycle induced by Palbociclib in ECs isolated from anti-BMP9/10-treated mice (FIG. 4B). Altogether, our data suggest that the induced late G1 state prevents the upregulation of genes responsible for the pathological metabolic rewiring associated with EC hyperproliferative state due to BMP9/10 signaling deficiency.

### Palbociclib Prevents Vascular Malformations in HHT Type 2.

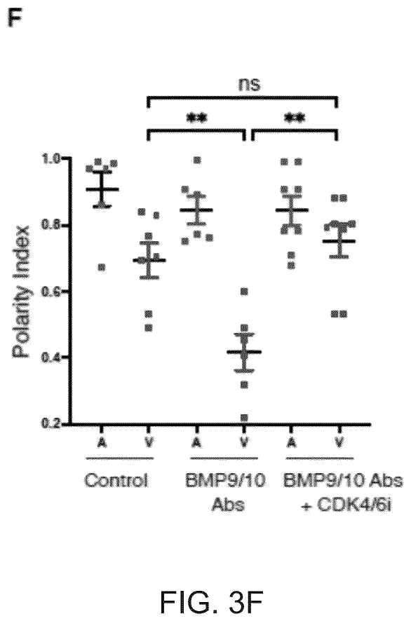

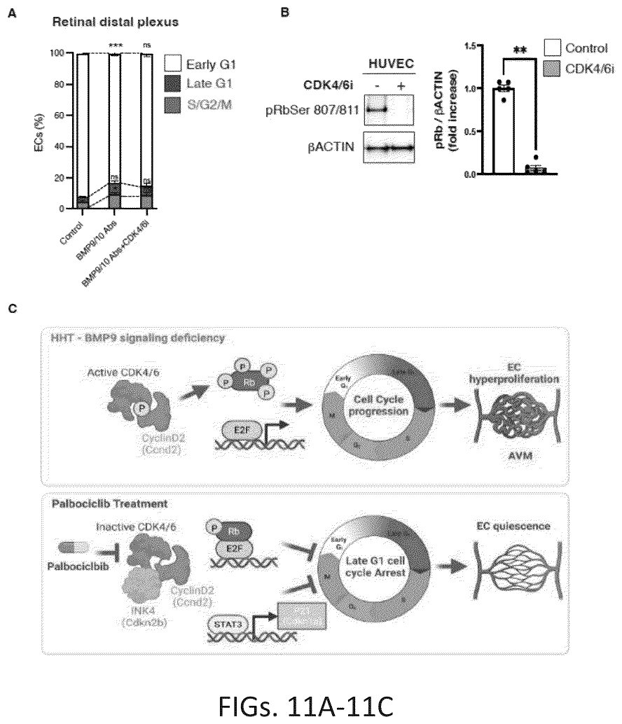

About 50% of HHT patients harbor mutations in the Acvrl1 gene¹, which encodes for the ALK1 receptor. To validate the clinical relevance of the use of Palbociclib for the treatment of HHT, we crossed Cdh5Cre^{ERT2} mice with Alk1^{flox/flox} mice (6-8-week-old) to generate an endothelial-specific inducible deletion of Alk1 (referred to as Alk1EC^{iKO}), which is an established preclinical mouse model of human HHT type 2^{12,25}. Neonatal Alk1EC^{iKO} pups received a single i.p. injection of tamoxifen at P3 to induce Alk1 gene deletion and were then treated with Palbociclib via oral gavage at P4, and their retinal vasculature was analyzed at P5 (FIG. 5A). While untreated Alk1EC^{iKO} animals developed numerous AV shunts, those that received Palbociclib exhibited a significantly lower number of AVMs (FIGS. 5B and C). Palbociclib treatment also prevented the formation of hyperdense and disorganized vascular networks at the front of the retinal vascular plexi in Alk1EC^{iKO} animals (FIGS. 5B and D) and normalized vascular progression (FIGS. 5B and E). Interestingly, unlike in the brains of mice treated with BMP9/10 blocking antibodies (FIGS. 10G and 10H), the brains of Alk1EC^{iKO} mice (FIG. 5F) exhibited significant dilations of the basilar and middle cerebral arteries (FIG. 13C), compared to control brains. In mice treated with a single dose of Palbociclib at P4, we observed a trend toward normalization of the diameter of the basilar and middle cerebral arteries (FIG. 5G-H). Due to the relatively short survival time post-tamoxifen induction in Alk1EC^{iKO} mice (approximately 48 hours), our study is limited to a single Palbociclib injection, which represents a technical challenge in exploring additional treatments. However, the number of latex-filled veins in Alk1EC^{iKO} brains treated with Palbociclib was significantly reduced when compared to untreated animals (FIG. 5I). In the lungs of Alk1EC^{iKO}, we observed strong latex blue dye extravasation across the intrapulmonary bed. In Alk1EC^{iKO} mice treated with Palbociclib, dye extravasation was reduced (FIG. 13A). Finally, as previously observed^{12,26}, in the intestinal tracts of P7 Alk1EC^{iKO} animals, we noticed the presence of veins filled with blue latex, demonstrating the presence of arteriovenous shunts in the gastrointestinal tract, that were absent in Alk1EC^{iKO} animals treated with CDK4/6i (FIG. 13B). These results demonstrate that Palbociclib prevents the development of vascular malformations in major organs affected in HHT patients, and more robustly effects brain AVMs induced by endothelial loss of Alk1 function. Single Cell RNA sequencing analysis of Palbociclib effects on endothelial arteriovenous identity in Alk1EC^{iKO} mice.

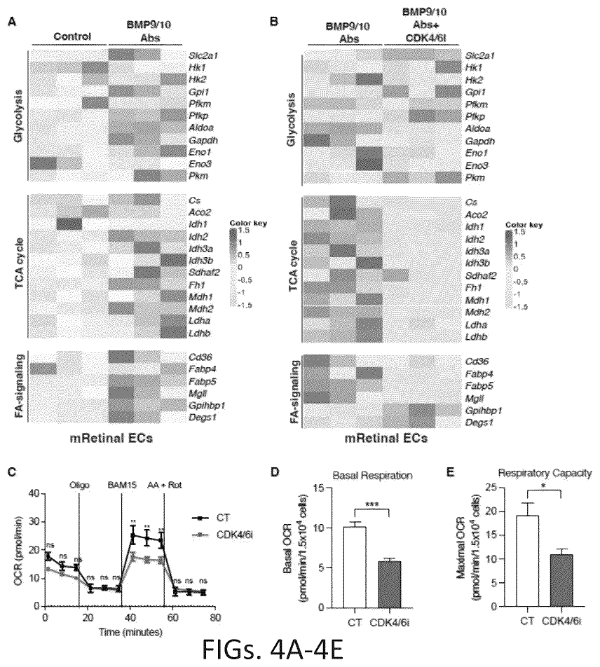

To gain a deeper understanding of the molecular mechanisms underlying the effects of Palbociclib on endothelial identity and cell cycle state, and vascular malformations in Alk1EC^{iKO} mice, we employed a single cell RNA sequencing (scRNAseq) approach. We isolated retinal ECs from P5 Alk1^{fl/fl}, Alk1^{fl/fl} treated with CDK4/6i, Alk1EC^{iKO}, and Alk1EC^{iKO} treated with CDK4/6i, animals (FIG. 6A). After retinal tissue dissociation, we FACS-isolated ECs (CD31⁺CD45⁻) that were used for scRNAseq analysis. After filtering, samples were processed for single cell barcoding and downstream mRNA library preparation and sequencing (FIG. 6B). Relative mRNA expression of genes known to be associated with arterial, venous, capillary, proliferative and tip cell identities were used to annotate the five EC populations (FIGS. 6C and D).

To investigate the effects of CDK4/6i on arteriovenous identity in HHT, we employed PHATE dimensionality reduction analysis. This method evaluates the relative gene expression levels among clusters within scRNAseq datasets to predict lineage relationships among the populations. As previously published by our group, we used arterial- or venous-enriched gene expression data to generate arterial and venous identity module “scores” for each EC cluster in the scRNAseq dataset⁸. Individual endothelial cell scores were then visualized on the PHATE plots (FIGS. 6E and G). As expected, in Alk1^{fl/fl} control ECs, cells in the arterial or venous clusters displayed a high arterial or venous score, respectively (FIGS. 6E and G). However, in retinal ECs from Alk1EC^{iKO}, both arterial and venous clusters had a low identity scoring, consistent with a loss of arteriovenous identity in HHT conditions (FIGS. 6E and G). More specifically, the mRNA expression of genes associated with arterial identity, including Efnb2, Gja4, Bmx, Jag1, and Unc5b, were downregulated in the arterial cluster (FIG. 6F); whereas, the mRNA expression of genes associated with venous identity were either downregulated such as Ephb4, Nr2f2, or upregulated such as Nrp2 and Emcn, in the venous cluster (FIG. 6H). In the Alk1EC^{iKO} animals treated with CDK4/6i, the arterial cluster presented a higher arterial score (FIG. 6E) and a higher mRNA expression of all genes associated with arterial identity compared to the Alk1EC^{iKO} group (FIG. 6F). Regarding the venous identity scoring and mRNA expression of venous genes in the venous cluster, we did not observe differences between Alk1EC^{iKO}, and Alk1EC^{iKO} treated with CDK4/6i groups (FIGS. 6G and H). In the Alk1^{fl/fl} animals treated with CDK4/6i, Palbociclib did not affect arterial or venous scoring nor the expression of arterial and venous genes in their respective clusters, compared to the Alk1^{fl/fl} group (FIG. 6E-H). These results demonstrate that, in the Alk1EC^{iKO} mouse model of HHT, at a single cell level, both arterial and venous identity of ECs are profoundly dysregulated. Palbociclib treatment prevents the downregulation of arterial-enriched genes in retinal ECs, whereas the expression of venous identity genes was not different within the timeframe of the experimental dosing.

### Analysis of Palbociclib Effects on Endothelial Cell Cycle State in Alk1EC^{iKO} Mice.

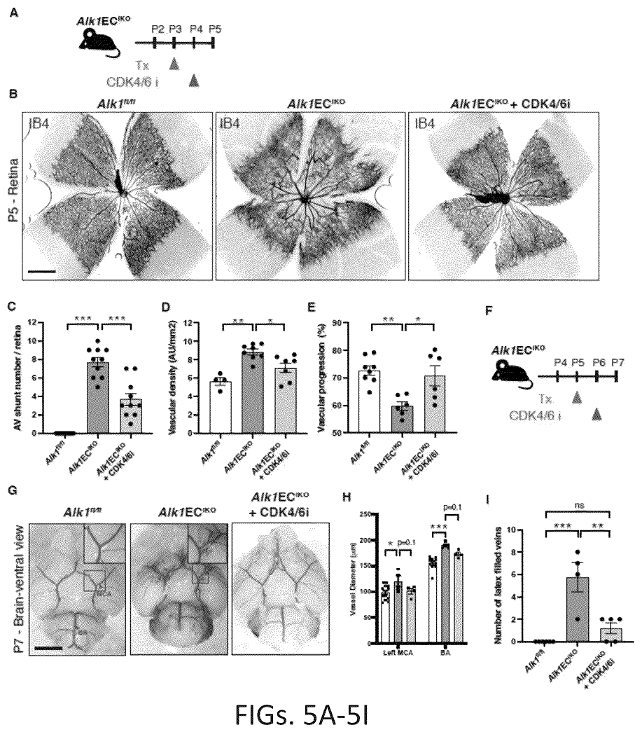

Since endothelial identities and cell cycle states are closely linked⁸, we further investigated the effects of Palbociclib on endothelial cell cycle state using Alk1EC^{iKO} mice expressing the Fucci2 reporter. Alk1EC^{iKO}-Fucci2 pups received a single i.p. injection of tamoxifen at P3 to induce Alk1 gene deletion and they were then treated with Palbociclib via oral gavage at P4, and their retinal vasculature was analyzed at P5 (FIG. 7A). In Alk1EC^{iKO} Fucci2 pups, we found a significant increase in ECs actively cycling in the S/G2/M phases in arteries, veins and capillaries, compared to controls (FIGS. 7B and C), consistent with the hyperproliferative state of ECs observed in HHT patients (FIG. 1E). However, in Alk1EC^{iKO} pups treated with CDK4/6i, the percentage of ECs residing in the S/G2/M phases was significantly lower in arteries and capillaries, concomitant with a higher proportion of ECs in late G1 state; no significant effects on venous ECs were observed (FIGS. 7B and C).

Additionally, using previously generated cell cycle state-specific gene expression data⁸, we generated and applied “early G1” and “late G1” cell cycle state scores to each EC in the scRNAseq dataset and visualized the results on PHATE plots (FIGS. 7D and E). In accordance with our previous studies⁸, in the Alk1^{fl/fl} group, the arterial cluster displayed a high late G1 score (FIG. 7D) and the venous cluster a high early G1 score (FIG. 7E). The late G1 score was lower in the arterial cluster of the Alk1EC^{iKO} group and was higher when Alk1EC^{iKO} animals were treated with CDK4/6i (FIG. 7D). These results are consistent with a loss of arterial identity in ECs from Alk1EC^{iKO} animals and a prevention of arterial identity loss in ECs from Alk1EC^{iKO} treated with CDK4/6i. Similarly, to the venous scoring in other analyses (FIG. 6G), the early G1 score in the venous cluster did not show any obvious differences between Alk1EC^{iKO} animals and Alk1EC^{iKO} animals treated with CDK4/6i (FIG. 7E). Similar results were also found when assessing venous endothelial cell cycle state in Alk1ECiKO-Fucci2 mice treated with CDKi4/6i (FIG. 7C).

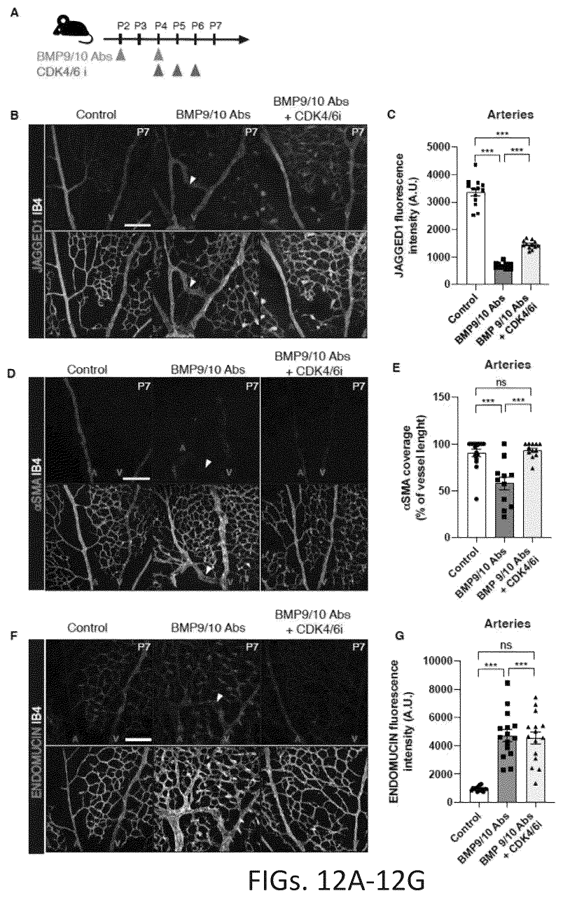

Finally, since we previously showed that the early G1 state was essential for BMP-induced venous genes, and late G1 state was essential for TGF-β1-induced arterial gene expression⁸, we evaluated the differential expression of key genes of the BMP and TGF-β pathways in the scRNAseq datasets. Correlating with a loss of arteriovenous identity in HHT, in Alk1EC^{iKO} ECs, we found lower expression of genes of the TGF-β pathway, such as Tgfbr1, Tgfbr2, Smad6 and Tgfb2 (FIG. 14A), as well as genes of the BMP pathway, including Eng (FIG. 14B), compared to control group. In ECs isolated from Alk1EC^{iKO} mice that received Palbociclib treatment, the expression of those genes was higher (FIGS. 14A and 14B).

### Palbociclib Inhibits VEGF-A Signaling and Promotes BMP9 Signaling.

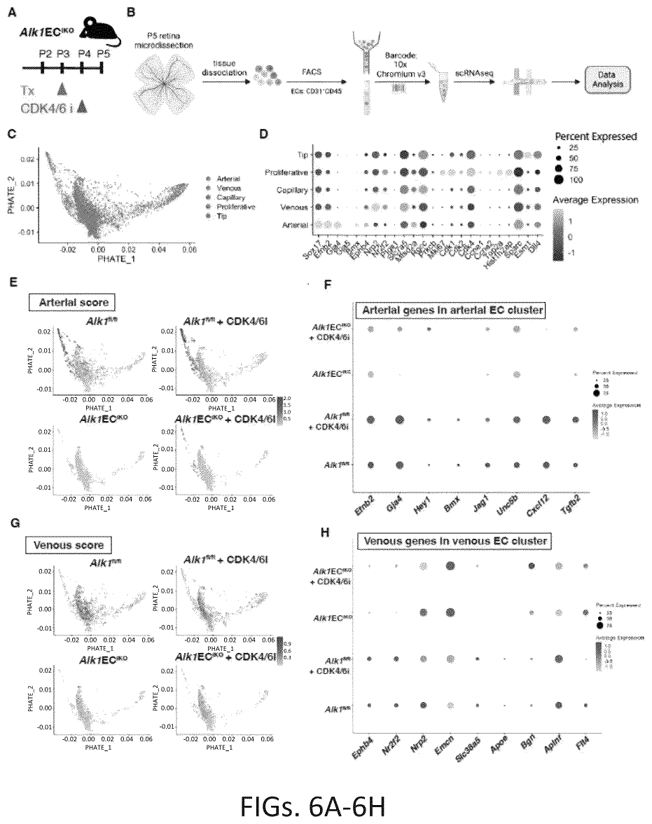

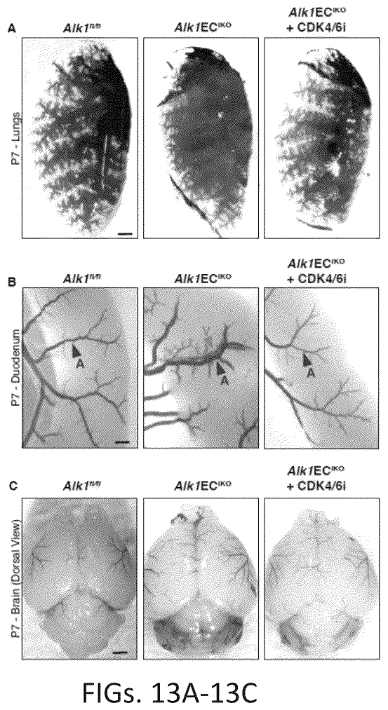

To decipher the molecular mechanism(s) underlying the effects of Palbociclib-induced endothelial cell cycle arrest on the prevention of AVMs, we tested the effects of CDK4/6 inhibition on VEGF-A and BMP9-mediated signaling, which are known to be dysregulated in HHT. As expected, Western Blot analysis revealed that a 15-minute VEGF-A stimulation of HUVECs leads to increased pAKT and pERK1/2 (FIGS. 8A and B). Interestingly, pre-treatment with Palbociclib for 24 h prior to VEGF-A stimulation significantly decreased pAKT and pERK1/2 activation. In addition, Palbociclib pre-treatment of HUVECs enhanced SMAD1/5/8 phosphorylation in response to BMP9 (FIGS. 8B and C). Next, we treated HUVECs with Alk1 siRNA to silence Alk1 and test the VEGF-A-induced response, with or without Palbociclib treatment, using pAKT activation as a readout. We did not detect any differences in the pAKT activation induced by VEGF-A between Alk1 silenced HUVECs treated, or not, with Palbociclib (FIGS. 15A and 15B). Consistent with this observation, the scRNAseq data did not show any obvious differences in the expression of genes associated with the AKT pathway between retinal ECs isolated from Alk1^{iECKO} mice treated, or not, with Palbociclib (FIG. 15C).

With bulk RNAseq analysis, we further investigated the effects of Palbociclib on these pathways in vivo on P7 retinal ECs from control pups or those treated with anti-BMP9/10, with or without CDK4/6i, as outlined in FIG. 2A. We found enhanced mRNA expression of several genes in the BMP9/10 signaling pathway, such as Eng, but also the anti-angiogenic gene Flt1, in pups treated with CDK4/6i, following anti-BMP9/10 treatment (FIG. 8D). In Alk1EC^{iKO} mice, we confirmed that Palbociclib promoted the expression of ENDOGLIN in venous ECs at both mRNA (FIG. 14B) and protein (FIGS. 8E and F) levels. We also investigated the potential role of FLT1, a decoy receptor for VEFG-A, in the mechanism of action of Palbociclib, by injecting i.p. FLT1 blocking antibodies in Alk1EC^{iKO} mice simultaneously with CDK4/6i treatment (FIG. 8G). We found, as expected, that Alk1^{iECKO} mice that were treated with Palbociclib developed less AV shunts than untreated Alk1^{iECKO} animals; however, Palbociclib-treated Alk1^{iECKO} mice that also received FLT1 antibodies exhibited numerous AV shunts, comparable to untreated Alk1^{iECKO} mice (FIGS. 8G and H). These results demonstrate that blocking FLT1 counteracts the effects of Palbociclib and emphasizes a mechanism of action inhibiting VEGF-A signaling via the decoy activity of VEGFR1/FLT1. Collectively, these results suggest that the cell cycle arrest, induced by Palbociclib, enables the expression of genes and proteins in the BMP9/10 signaling pathway that help to restore this defective signaling axis in HHT and prevent overactivation of proangiogenic signaling.

### Discussion

The present study reveals that endothelial cell cycle states are dysregulated during the pathogenesis of vascular malformations in preclinical models of HHT, as well as in human dermal telangiectases from HHT type 2 patients. We also showed the clinical relevance of Palbociclib in the prevention of AVMs in a preclinical model of HHT type 2. Mechanistically, late G1 cell cycle state induced by CDK4/6 inhibition enables the expression of genes regulating VEGF-A and BMP9 signaling, EC proliferation, migration and metabolism that collectively contribute to the prevention of vascular malformations induced by BMP9/10 immunosuppression or endothelial-specific Alk1 gene deletion.

Several studies have shown that EC hyperproliferation and loss of arteriovenous identity are key characteristics of vascular malformations in HHT models^{12,27,28}. However, the mechanistic link between endothelial cell cycle dysregulation and AVM development was lacking. Our group previously reported that, during normal vascular development, endothelial cell cycle state is a critical regulator of arteriovenous identity. Herein, we show that during HHT pathogenesis, venous- and arterial-specific cell cycle states are profoundly disturbed. We found that this change in endothelial cell cycle state is associated with the dysregulation of mRNA expression of genes that regulate cell cycle progression and endothelial arteriovenous identity that are likely contributing to AVM development. It is well understood that BMP9/10 signaling deficiency leads to an overactivation of proangiogenic pathways controlled by VEGF-A^{4,29}, resulting in impaired EC proliferation control, migration, and permeability. However, the exact molecular mechanism(s) triggering ECs to progress through the cell cycle in response to BMP9/10 signaling deficiency was unknown. Our work uncovered that Cond2 and Cdk6, key genes promoting cell cycle checkpoint progression are upregulated in HHT conditions.

In the present study, re-purposing the oral drug Palbociclib (CDK4/6i), currently used in combination with endocrine therapy for the treatment of metastatic breast cancer^{30,31} showed relevance for the prevention of AVM formation in neonatal preclinical models of HHT. Further studies investigating the clinical relevance of Palbociclib in the treatment of established AVMs in human patients are needed. Although this work focused primarily on understanding the role of endothelial cell cycle regulation in AVM formation, blood vessels are also comprised of mural cells (SMCs and pericytes) that are known to be altered in Alk1EC^{iKO}, EngEC^{iKO} and SMAD4EC^{iKO} HHT mouse models^{12,32}. We showed that CDK4/6 inhibition normalizes SMC vessel coverage in the vascular plexi of animals treated with BMP9/10 antibodies. However, whether this is due to direct effects of Palbociclib on SMCs or indirect consequences of improved vascular remodeling due to late G1 induction in ECs needs to be determined.

To date, the therapeutic options available for HHT patients are intended to reduce the symptoms of the disease, such as epistaxis⁴. However, preclinical, and clinical studies using anti-VEGF-A, anti-ANGT2 molecules and PI3K inhibitors are emerging to counterbalance the pro-angiogenic axis over-activated in HHT and, ultimately, correct AVMs to a normal vasculature^{12,33-36}. In addition, instead of targeting the pro-angiogenic signals, current studies are now aiming to restore the defective BMP9-ALK1-SMAD signaling axis in HHT, using the mTOR signaling inhibitors, such as Sirolimus, that block the PI3K signaling pathway that is overactivated in HHT1 and HHT2^{14,37}. Another therapeutic strategy is to promote ALK1-mediated signaling with Tacrolimus, leading to beneficial clinical effects on vascular malformations related to HHT³⁸. In the two preclinical models of HHT used in this study, we demonstrated that endothelial cell cycle arrest induced by Palbociclib enables the expression of Eng, probably participating in the reestablishment of the deficient BMP9 signaling pathway. However, in the context of Alk1 depletion, another study showed that Eng overexpression in Alk1^{ECiKO} mice does not prevent AVM formation²⁸. Therefore, further studies are necessary to fully address the impact of Palbociclib on endothelial ALK1/ENDOGLIN/SMAD signaling. Nevertheless, upregulation of the antiangiogenic gene Flt1 induced by CDK4/6 inhibition, also appears to counterbalance the overactivated VEGF-A pathway observed in ECs in HHT.

Furthermore, we observed phenotypic differences in the two postnatal mouse models of HHT used in our studies (BMP9/10 Abs vs. Alk1^{ECiKO} mouse). While the genetic deletion of Alk1 (Alk1^{ECiKO}) induces vascular malformations in various organs^{12,26}, we found that the pharmacological approach using BMP9/10 blocking antibodies leads to vascular anomalies only in vascular beds where active vascular remodeling occurs post-natally,^{39,40} such as the retina and lungs. Also, the differences could be linked to the differential expression levels of BMP9 signaling in different organs. It is known that BMP9 plays an important role in regulating biological functions and tissue homeostasis of the lungs and retinas. In 2-week-old mice, BMP9 expression is higher in the lungs than in the brain⁴¹. This observation might explain why we found a striking phenotype in the lungs of BMP9/10 Abs-treated mice but not in the brains of these animals.

Several studies suggested that defective blood flow and/or altered EC migration contributes to the biogenesis of vascular malformations^{17,27,42}. Our group previously showed that in physiological vascular development, flow shear forces regulate endothelial cell identity via the cell cycle regulator p27¹⁶. Here, in HHT, we demonstrated that aberrant endothelial cell cycle control leads to non-polarized endothelial cell migration, and that induced late G1 arrest normalizes flow-mediated endothelial cell polarization. However, in vascular malformations, whether disrupted blood flow forces are upstream of aberrant endothelial cell cycle control and/or migration is still unaddressed.

Endothelial metabolic perturbations have been implicated in the pathogenesis of many cardiovascular diseases²³. Herein, we found that a profound dysregulation of a large spectrum of genes involved in the regulation of endothelial metabolism in HHT. Indeed, we found that BMP9/10 immunosuppression induced an overexpression of genes involved in glycolysis, TCA cycle and fatty acid metabolic pathways. This observation is consistent with the idea that a metabolic rewiring happens when ECs are overactivated (i.e., hyperproliferative) under pathological conditions to adapt to their new environmental conditions, such as increased flow shear forces or hypoxia. Whether theses metabolic changes associated with vascular malformations are the consequences or causes of endothelial cell cycle state dysregulation still needs to be determined. Nevertheless, endothelial late G1 cell cycle state induced by Palbociclib in BMP9/10 antibody-treated mice was found to be associated with decreased expression of metabolic genes in ECs correlated with a normalization of the vasculature, suggesting that cell cycle state modulation might be upstream of metabolic rewiring.

In conclusion, this study provides new insights into molecular mechanisms leading to HHT by defining how endothelial cell cycle is dysregulated in AVMs due to BMP9/10 and Alk1 signaling deficiencies. It also shows that cell cycle modulators, such as Palbociclib, may represent new options for the treatment of vascular malformations in HHT patients. Since dysregulation of endothelial cell cycle control is certainly not restricted to HHT, the present work also opens new therapeutic strategies for a larger group of diseases characterized by endothelial cell hyperproliferation and loss of identity, such as Cerebral Cavernous Malformations and/or Venous Malformations.

### LITERATURE CITED

1. Shovlin C L. Hereditary haemorrhagic telangiectasia: pathophysiology, diagnosis and treatment. Blood Rev. 2010; 24:203-19.
2. Uebelhoer M, Boon L M and Vikkula M. Vascular anomalies: from genetics toward models for therapeutic trials. Cold Spring Harb Perspect Med. 2012; 2.
3. Marziano C, Genet G and Hirschi K K. Vascular endothelial cell specification in health and disease. Angiogenesis. 2021; 24:213-236.
4. Robert F, Desroches-Castan A, Bailly S, Dupuis-Girod S and Feige J J. Future treatments for hereditary hemorrhagic telangiectasia. Orphanet J Rare Dis. 2020; 15:4.
5. Tual-Chalot S, Oh S P and Arthur H M. Mouse models of hereditary hemorrhagic telangiectasia: recent advances and future challenges. Front Genet. 2015; 6:25.
6 Foster D A, Yellen P, Xu L and Saqcena M. Regulation of G1 Cell Cycle Progression:
Distinguishing the Restriction Point from a Nutrient-Sensing Cell Growth Checkpoint(s). Genes Cancer. 2010; 1:1124-31.
7. Saqcena M, Menon D, Patel D, Mukhopadhyay S, Chow V and Foster D A. Amino acids and mTOR mediate distinct metabolic checkpoints in mammalian G1 cell cycle. PLOS One. 2013; 8:e74157.
8 Chavkin N W, Genet G, Poulet M, Jeffery E D, Marziano C, Genet N, Vasavada H, Nelson E A, Acharya B R, Kour A, Aragon J, McDonnell S P, Huba M, Sheynkman G M, Walsh K and Hirschi K K. Endothelial cell cycle state determines propensity for arterial-venous fate. Nat Commun. 2022; 13:5891.
9. Sakaue-Sawano A, Kurokawa H, Morimura T, Hanyu A, Hama H, Osawa H, Kashiwagi S, Fukami K, Miyata T, Miyoshi H, Imamura T, Ogawa M, Masai H and Miyawaki A. Visualizing spatiotemporal dynamics of multicellular cell-cycle progression. Cell. 2008; 132:487-98.
10. Fry D W, Harvey P J, Keller P R, Elliott W L, Meade M, Trachet E, Albassam M, Zheng X, Leopold W R, Pryer N K and Toogood P L. Specific inhibition of cyclin-dependent kinase 4/6 by PD 0332991 and associated antitumor activity in human tumor xenografts. Mol Cancer Ther. 2004; 3:1427-38.
11. Gallanis G T, Pericas R I, Riegel A T and Pohlmann P R. An evaluation of palbociclib as a breast cancer treatment option: a current update. Expert Opin Pharmacother. 2021; 22:281-290.
12. Ola R, Dubrac A, Han J, Zhang F, Fang J S, Larrivee B, Lee M, Urarte A A, Kraehling J R, Genet G, Hirschi K K, Sessa W C, Canals F V, Graupera M, Yan M, Young L H, Oh P S and Eichmann A. PI3 kinase inhibition improves vascular malformations in mouse models of hereditary haemorrhagic telangiectasia. Nat Commun. 2016; 7:13650.
13. Ruiz S, Zhao H, Chandakkar P, Chatterjee P K, Papoin J, Blanc L, Metz C N, Campagne F and Marambaud P. A mouse model of hereditary hemorrhagic telangiectasia generated by transmammary-delivered immunoblocking of BMP9 and BMP10. Sci Rep. 2016; 5:37366.
14. Iriarte A, Figueras A, Cerda P, Mora J M, Jucgla A, Penin R, Vinals F and Riera-Mestre A. PI3K (Phosphatidylinositol 3-Kinase) Activation and Endothelial Cell Proliferation in Patients with Hemorrhagic Hereditary Telangiectasia Type 1. Cells. 2019; 8.
15. Kim S, Leong A, Kim M and Yang H W. CDK4/6 initiates Rb inactivation and CDK2 activity coordinates cell-cycle commitment and G1/S transition. Sci Rep. 2022; 12:16810.
16. Fang J S, Coon B G, Gillis N, Chen Z, Qiu J, Chittenden T W, Burt J M, Schwartz M A and Hirschi K K. Shear-induced Notch-Cx37-p27 axis arrests endothelial cell cycle to enable arterial specification. Nat Commun. 2017; 8:2149.
17. Park H, Furtado J, Poulet M, Chung M, Yun S, Lee S, Sessa W C, Franco C A, Schwartz M A and Eichmann A. Defective Flow-Migration Coupling Causes Arteriovenous Malformations in Hereditary Hemorrhagic Telangiectasia. Circulation. 2021; 144:805-822.
18. Lee H W, Xu Y, He L, Choi W, Gonzalez D, Jin S W and Simons M. Role of Venous Endothelial Cells in Developmental and Pathologic Angiogenesis. Circulation. 2021; 144:1308-1322.
19. Genet G, Boye K, Mathivet T, Ola R, Zhang F, Dubrac A, Li J, Genet N, Henrique Geraldo L, Benedetti L, Kunzel S, Pibouin-Fragner L, Thomas J L and Eichmann A. Endophilin-A2 dependent VEGFR2 endocytosis promotes sprouting angiogenesis. Nat Commun. 2019; 10:2350.
20. Rama N, Dubrac A, Mathivet T, Ni Charthaigh R A, Genet G, Cristofaro B, Pibouin-Fragner L, Ma L, Eichmann A and Chedotal A. Slit2 signaling through Robo1 and Robo2 is required for retinal neovascularization. Nat Med. 2015; 21:483-91.
21. Dubrac A, Genet G, Ola R, Zhang F, Pibouin-Fragner L, Han J, Zhang J, Thomas J L, Chedotal A, Schwartz M A and Eichmann A. Targeting NCK-Mediated Endothelial Cell Front-Rear Polarity Inhibits Neovascularization. Circulation. 2016; 133:409-21.
22. Franco C A, Jones M L, Bernabeu M O, Geudens I, Mathivet T, Rosa A, Lopes F M, Lima A P, Ragab A, Collins R T, Phng L K, Coveney P V and Gerhardt H. Dynamic endothelial cell rearrangements drive developmental vessel regression. PLOS Biol. 2015; 13:e1002125.
23. Falkenberg K D, Rohlenova K, Luo Y and Carmeliet P. The metabolic engine of endothelial cells. Nat Metab. 2019; 1:937-946.
24. Luo W, Garcia-Gonzalez I, Fernandez-Chacon M, Casquero-Garcia V, Sanchez-Munoz M S, Muhleder S, Garcia-Ortega L, Andrade J, Potente M and Benedito R. Arterialization requires the timely suppression of cell growth. Nature. 2021; 589:437-441.
25. Tual-Chalot S, Mahmoud M, Allinson K R, Redgrave R E, Zhai Z, Oh S P, Fruttiger M and Arthur H M. Endothelial depletion of Acvrl1 in mice leads to arteriovenous malformations associated with reduced endoglin expression. PLOS One. 2014; 9:e98646.
26. Park S O, Wankhede M, Lee Y J, Choi E J, Fliess N, Choe S W, Oh S H, Walter G, Raizada M K, Sorg B S and Oh S P. Real-time imaging of de novo arteriovenous malformation in a mouse model of hereditary hemorrhagic telangiectasia. J Clin Invest. 2009; 119:3487-96.
27. Ola R, Kunzel S H, Zhang F, Genet G, Chakraborty R, Pibouin-Fragner L, Martin K, Sessa W, Dubrac A and Eichmann A. SMAD4 Prevents Flow Induced Arteriovenous Malformations by Inhibiting Casein Kinase 2. Circulation. 2018; 138:2379-2394.
28. Hwan Kim Y, Vu P N, Choe S W, Jeon C J, Arthur H M, Vary C P H, Lee Y J and Oh S P. Overexpression of Activin Receptor-Like Kinase 1 in Endothelial Cells Suppresses Development of Arteriovenous Malformations in Mouse Models of Hereditary Hemorrhagic Telangiectasia. Circ Res. 2020; 127:1122-1137.
29. Garcia de Vinuesa A, Abdelilah-Seyfried S, Knaus P, Zwijsen A and Bailly S. BMP signaling in vascular biology and dysfunction. Cytokine Growth Factor Rev. 2016; 27:65-79.
30. Serra F, Lapidari P, Quaquarini E, Tagliaferri B, Sottotetti F and Palumbo R. Palbociclib in metastatic breast cancer: current evidence and real-life data. Drugs Context. 2019; 8:212579.
31. Tripathy D, Blum J L, Rocque G B, Bardia A, Karuturi M S, Cappelleri J C, Liu Y, Zhang Z, Davis K L and Wang Y. POLARIS: a prospective, multicenter, noninterventional study assessing palbociclib in hormone receptor-positive advanced breast cancer. Future Oncol. 2020; 16:2475-2485.
32. Mahmoud M, Allinson K R, Zhai Z, Oakenfull R, Ghandi P, Adams R H, Fruttiger M and Arthur H M. Pathogenesis of arteriovenous malformations in the absence of endoglin. Circ Res. 2010; 106:1425-33.
33. Flieger D, Hainke S and Fischbach W. Dramatic improvement in hereditary hemorrhagic telangiectasia after treatment with the vascular endothelial growth factor (VEGF) antagonist bevacizumab. Ann Hematol. 2006; 85:631-2.
34. Kovacs-Sipos E, Holzmann D, Scherer T and Soyka M B. Nintedanib as a novel treatment option in hereditary haemorrhagic telangiectasia. BMJ Case Rep. 2017; 2017.
35. Faughnan M E, Gossage J R, Chakinala M M, Oh S P, Kasthuri R, Hughes C C W, McWilliams J P, Parambil J G, Vozoris N, Donaldson J, Paul G, Berry P and Sprecher D L. Pazopanib may reduce bleeding in hereditary hemorrhagic telangiectasia. Angiogenesis. 2019; 22:145-155.
36 Crist A M, Zhou X, Garai J, Lee A R, Thoele J, Ullmer C, Klein C, Zabaleta J and Meadows S M. Angiopoietin-2 Inhibition Rescues Arteriovenous Malformation in a Smad4 Hereditary Hemorrhagic Telangiectasia Mouse Model. Circulation. 2019; 139:2049-2063.
37. Iriarte A, Ochoa-Callejero L, Garcia-Sanmartin J, Cerda P, Garrido P, Narro-Iniguez J, Mora-Lujan J M, Jucgla A, Sanchez-Corral M A, Cruellas F, Gamundi E, Ribas J, Castellote J, Vinals F, Martinez A and Riera-Mestre A. Adrenomedullin as a potential biomarker involved in patients with hereditary hemorrhagic telangiectasia. Eur J Intern Med. 2021; 88:89-95.
38. Ruiz S, Chandakkar P, Zhao H, Papoin J, Chatterjee P K, Christen E, Metz C N, Blanc L, Campagne F and Marambaud P. Tacrolimus rescues the signaling and gene expression signature of endothelial ALK1 loss-of-function and improves HHT vascular pathology. Hum Mol Genet. 2017; 26:4786-4798.
39. Gao Y and Raj J U. Regulation of the pulmonary circulation in the fetus and newborn. Physiol Rev. 2010; 90:1291-335.
40. Sun Y and Smith L E H. Retinal Vasculature in Development and Diseases. Annu Rev Vis Sci. 2018; 4:101-122.
41. Liu W, Deng Z, Zeng Z, Fan J, Feng Y, Wang X, Cao D, Zhang B, Yang L, Liu B, Pakvasa M, Wagstaff W, Wu X, Luo H, Zhang J, Zhang M, He F, Mao Y, Ding H, Zhang Y, Niu C, Haydon R C, Luu H H, Wolf J M, Lee M J, Huang W, He T C and Zou Y. Highly expressed BMP9/GDF2 in postnatal mouse liver and lungs may account for its pleiotropic effects on stem cell differentiation, angiogenesis, tumor growth and metabolism. Genes Dis. 2020; 7:235-244.
42. Baeyens N, Larrivee B, Ola R, Hayward-Piatkowskyi B, Dubrac A, Huang B, Ross T D, Coon B G, Min E, Tsarfati M, Tong H, Eichmann A and Schwartz M A. Defective fluid shear stress mechanotransduction mediates hereditary hemorrhagic telangiectasia. J Cell Biol. 2016; 214:807-16.
43. Sorensen I, Adams R H and Gossler A. DLL1-mediated Notch activation regulates endothelial identity in mouse fetal arteries. Blood. 2009; 113:5680-8.
44. Abe T, Sakaue-Sawano A, Kiyonari H, Shioi G, Inoue K, Horiuchi T, Nakao K, Miyawaki A, Aizawa S and Fujimori T. Visualization of cell cycle in mouse embryos with Fucci2 reporter directed by Rosa26 promoter. Development. 2013; 140:237-46.
45 Shovlin C L, Angus G, Manning R A, Okoli G N, Govani F S, Elderfield K, Birdsey G M, Mollet I G, Laffan M A and Mauri F A. Endothelial cell processing and alternatively spliced transcripts of factor VIII: potential implications for coagulation cascades and pulmonary hypertension. PLOS One. 2010; 5:e9154.
46. Chavkin N W, Cain S, Walsh K and Hirschi K K. Isolation of Murine Retinal Endothelial Cells for Next-Generation Sequencing. J Vis Exp. 2021.
47. Blankenberg D, Gordon A, Von Kuster G, Coraor N, Taylor J, Nekrutenko A and Galaxy T. Manipulation of FASTQ data with Galaxy. Bioinformatics. 2010; 26:1783-5.
48. Blankenberg D, Von Kuster G, Coraor N, Ananda G, Lazarus R, Mangan M,
Nekrutenko A and Taylor J. Galaxy: a web-based genome analysis tool for experimentalists. Curr Protoc Mol Biol. 2010; Chapter 19:Unit 19 10 1-21.
49. Dobin A, Davis C A, Schlesinger F, Drenkow J, Zaleski C, Jha S, Batut P, Chaisson M and Gingeras T R. STAR: ultrafast universal RNA-seq aligner. Bioinformatics. 2013; 29:15-21.
50. Love M I, Huber W and Anders S. Moderated estimation of fold change and dispersion for RNA-seq data with DESeq2. Genome Biol. 2014; 15:550.
51. Hafemeister C and Satija R. Normalization and variance stabilization of single-cell RNA-seq data using regularized negative binomial regression. Genome Biol. 2019; 20:296.

### Materials and Methods for Example 1

### Animals

All animal protocols and procedures were approved by the University of Virginia Animal Care and Use Committee and complied with all ethical regulations. Cdh5Cre^{ERT2}1Rha⁴³ and Alk1^{flox/flox} were gifted from Drs. Ralf Adams and Paul Oh, respectively. R26p-Fucci2 (Fucci2) (Accession #CDB0203T)⁴⁴ and C57bl/6 mice were purchased from Riken and Jax, respectively. Cdh5Cre^{ERT2} mice were crossed with Alk1^{flox/flox} mice (6-8-week-old) to generate Alk1EC^{iKO} pups. Cre activity in Alk1EC^{iKO} was induced in pups at P3 via a single intraperitoneal (i.p.) injection of Tamoxifen (Tx; Sigma T5648, 25 ml/day of 4 mg/ml, resuspended in 10% ethanol and 90% corn oil). Alk1^{flox/flox} littermates were used as controls. Alk1EC^{iKO} mice were crossed with Fucci2 mice to generate Alk1EC^{iKO}-Fucci2 pups. For studies using mouse monoclonal anti-BMP9 (IgG2b, MAB3209, R&D, 10 mg/kg in PBS1X) and anti-BMP10 (IgG2a, MAB2926, R&D, 10 mg/kg in PBS1X) antibodies (BMP9/10 Abs), pups received i.p. injections of BMP9/10 Abs at P2 and P4 and were sacrificed at P7. For Palbociclib treatment (CDK4/6 inhibitor, CDK4/6i; Sigma, PZ083 resuspended in 50 mM sodium lactate), pups received oral gavage as reported by our group⁸ (50 ml/day of 12 mg/ml, 120 mg/kg/d) starting on the second day of BMP9/10 Abs injection at P4, P5 and P6 and were sacrificed at P7. For FLT1 Abs treatment (R&D, MAB471), a single i.p. injection of FLT1 Abs (1.6 mg/kg) simultaneously with CDK4/6i treatment in Alk1^{iECKO} mice P5.

### Genotyping

DNA was isolated from tail clippings and lysed in alkaline lysis reagent (pH 12) (25 mM NaOH and 0.2 mM EDTA) for 1 h at 90° C. (hotshot method) and neutralized with 40 mM Tris-HCl. PCR was performed in PCR-ready tubes (Bioneer Inc. K-2016) with the following primers.

TABLE 1
PCR genotyping primers sequences

| | |
|---|---|
| Cdh5Cre^{ERT2} | Forward sequence (5′-3′): |
| | AATCTCCCACCGTCAGTACG |
| | (SEQ ID # 1) |
| | Reverse sequence (5′-3′): |
| | CGTTTTCTGAGCATACCTGGA |
| | (SEQ ID # 4) |
| Alk1^{flox/fox} | Forward sequence (5′-3′): |
| | CAGCACCTACATCTTGGGTGGAGA |
| | (SEQ ID # 2) |
| | Reverse sequence (5′-3′): |
| | ACTGTTCTTCCTCGGAGCCTTGTC |
| | (SEQ ID # 3) |

### Patient Inclusion

Patients were selected from the referral HHT Unit at the Hospital Universitari de Bellvitge (Barcelona, Spain). Patients from were considered eligible for enrolment if they were over 18 years, meeting a definite diagnosis according to Curaçao Criteria, showing mandatory cutaneous telangiectasia on the fingertip, and had a positive genetic study for Eng or Avcrl1⁴⁵. All selected patients gave their signed informed consent for skin biopsy in accordance with local Clinical Research Ethics Committee requirements. Three HHT2 patients and three controls were included. Cutaneous telangiectasia biopsies and control samples from healthy skin in resection borders from patients with melanoma were collected. Briefly, a punch biopsy (3 mm) from a cutaneous telangiectasia on the fingertip of each patient was obtained under sterile conditions. Biopsy samples were encrypted according to a code assigned to each patient and fixed in buffered formalin, dehydrated, and embedded in paraffin.

### Immunochemistry on Human Skin Samples

Tissue sections (3 μm) were stained by immunohistochemistry to determine the amount of expression of Ki67. Samples were deparaffined in xylene, rehydrated in downgraded alcohols, and distilled water. Antigen retrieval was performed under high-pressure conditions for 3 or 4 min in citrate buffer, pH 6 or 6.5, and incubated with 3% H2O2 for 10 min. Samples were then blocked with 1:20 goat serum for 1 h followed by incubation overnight at 4° C. with monoclonal rabbit anti-Ki-67 (SP6, Epredia Netherlands B.V.). Sections were incubated with the specific secondary antibodies (VECTON Immpress), followed by the DAB developing system (VECTON). Samples were counterstained with hematoxylin and eosin, and visualized under light microscopy.

### Retina Immunostaining and Image Analysis

Eyes from P7 pups were pre-fixed in 4% PFA for 20 min at room temperature (RT). The retinas were dissected out and permeabilized for 30 min at RT in 0.5% Triton X-100 and then incubated with antibodies in retina buffer (1% FBS, 3% BSA and 0.5% Triton X-100 in PBS1x) overnight at 4° C. Retinas were washed with PBS1X and incubated with secondary antibodies in retina buffer for 2 h at RT. Retinas were washed with PBS1X, mounted with DAKO mounting medium (DAKO) and imaged using confocal microscopy (LEICA SP8 and LASX software). Quantification of vascular progression, density, AVM numbers and vessel diameters in the retinal vasculature were performed with Image J software.

### Bulk RNA Sequencing of Primary Mouse Retinal Endothelial Cells

Eyes from P7 pups of control (group 1), BMP9/10 Abs-treated (group 2), and BMP9/10 Abs+CDK4/6i (group 3) treated animals were dissected out and primary retinal ECs were FACS-isolated and prepared for bulk RNA sequencing as previously described^{8,46}. Six to nine retinas/group were pooled for 1 experiment and each experiment was repeated 3 times (N=3). mRNA samples from retinal ECs from groups 1, 2 and 3 were isolated using RNeasy Plus Micro Kit (QIAGEN, cat #74034). Next-generation whole transcriptome Illumina sequencing (HiSeq4000) was performed by the Yale Center for Genome Analysis. The raw Fastq-files were generated with bcl2fastq2_v2.19.0, and quality control checks were done using FastQC software^{47,48}. The reads were mapped to the mouse reference genome “GRCm39” and alignments and quantification of mRNA was performed with the STAR aligner⁴⁹. Differential expression analysis was done with DESeq2 in R⁵⁰, and considered significant genes based on the p-adjusted values (Benjamini and Hochberg method for controlling FDR) of less than or equal to 0.05.

### Library Preparation and Sequencing

Retinal endothelial cells from P5 Alk1^{fl/fl}, Alk1^{fl/fl} treated with CDK4/6i, Alk1EC^{iKO}, and Alk1EC^{iKO} treated with CDK4/6i animals were isolated using a previously described and published method optimized for purification and viability⁷². Briefly, dissected retinas were digested with Collagenase Type II (1.0 mg/mL, Gibco Cat #17101015) in DMEM (Gibco Cat #21013024) and 10% FBS (Gibco Cat #26140079) for 20 min, washed, immunolabeled with anti-CD31 and anti-CD45 in staining buffer (HBSS with 10% FBS, 20 mM HEPES, 1 mg/mL D-Glucose), then resuspended in FACS buffer (PBS with 1% FBS). FACS-purified live single cell suspensions were submitted to the University of Virginia Genome Analysis and Technology Core (RRID:SCR_018883) for single cell RNA library preparation and next-generation sequencing. Single cell RNA libraries were prepared using the 10× Genomics Chromium Next GEM Single Cell 3′ Reagent Kit v3.1 (PN-1000121) and Chromium Controller. Next-generation sequencing was performed using an Illumina NextSeq 2000 Sequencing System on the P3 flow cell with a 100 bp paired-end sequencing reagent kit. Estimated cell number outputs for each sample were: Alk1^{fl/fl}=1535; Alk1^{fl/fl}+CDK4/6i=1535; Alk1EC^{iKO}=1535; Alk1EC^{iKO}+CDK4/6i=1535. The scRNAseq data were processed using the Cellranger (v7.0) count pipeline. This involved aligning the fastq files with the mouse reference transcriptome (mm10), performing filtering, and counting barcodes/UMIs. The output of the Cellranger pipeline, which included filtered feature-barcode matrices, was then analyzed using the Seurat v4.3 package in R. Data was filtered to include features that were detected in at least three cells, and cells were selected based on their expression of at least 200 features. Quality control metrics were applied to eliminate cells that might be dying/dead, possible doublets, and cells with high mitochondrial gene expression (3-8% of total transcripts) for all four experimental conditions. The data were normalized using the improved “sctransform” method⁵¹. To remove the effects of cell cycle phase differences among proliferating cells alone while preserving signals separating non-cycling and cycling cells, the difference between the G2M and S phase cell cycle scores was regressed out. Data integration methods were used to match or anchor cell populations that were shared across all four experimental datasets. This allowed for comparative analysis across all experimental conditions and corrected for batch effects. Principal Component Analysis (PCA) was performed, followed by finding nearest neighbors using PCA dimensions 1 to 16, and clustering was done at a resolution of 0.7. UMAP (Uniform Manifold Approximation and Projection), and these data were used for further dimensionality reduction. Cell types were identified based on known markers and marker analysis using Seurat. The scaled data from the integrated assay of the Seurat object was used to create and plot the high-dimension manifold of the data using PHATE (Potential of Heat-diffusion for Affinity-based Trajectory Embedding) with parameters knn=10 and t=32. Downstream marker analysis was performed using the normalized “RNA assay” for comparisons across all experimental conditions. Module scores for arterial-(late G1) and venous-(early G1) enriched genes were calculated for each cell. To ensure fair comparison, an equal number of cells were analyzed across all experimental conditions by down sampling cells to the lowest number of cells in an experimental condition.

### Blue Latex Dye Perfusion

P7 pups were deeply anesthetized with isoflurane using a nose cone and a thoracotomy was performed to expose the heart. For brain and gastrointestinal vasculature perfusions, the right atrium was cut and an insulin syringe (27 G) pre-filled with 1 ml of blue latex (VWR 470024-612) was inserted in the apex of the left ventricle to perfuse approximately 600 ml of latex. For lung vasculature perfusion, the left atrium was cut and an insulin syringe (27 G) pre-filled with 1 ml of blue latex was inserted in the right ventricle to perfuse approximately 300 ml of latex. Brains, lungs, and gastrointestinal tracts were dissected out, washed in PBS1X and post-fixed with 4% PFA overnight. After, post-fixation, lungs were dehydrated with methanol (30%, 60% and 100% successively for 24 h each). Post-dehydration, were clarified with Benzyl Benzoate:Benzyl alcohol (1:1) for at least 48 h Imaging was done using a Nikon SMZ-745T Trinocular 4K Digital Stereo microscope.

### Cell Culture

Primary Human Umbilical Vein Endothelial Cells (HUVECs) were purchased from Lonza (C2519AS) and cultured in EGM™2-Bulletkit™ medium (CC-3156 & CC-4176, Lonza). For VEGF-A- or BMP9-induced signaling response in HUVECs, cells (70% confluent) were treated with Palbociclib (3 mg/ml, CDK4/6i) for 12 h in EGM-2 medium prior to starvation overnight in EMB-2 media (CC-3162 Lonza) supplemented with 0.1% FBS and Palbociclib (3 mg/ml). Cells were stimulated with either VEGF-A (25 ng/ml for 15 min) or BMP9 (1 ng/ml for 2 h) and harvested for Western Blot analysis.

siRNA Transfection

Alk1 siRNAs (Hs_ACVRL1_6, SI02758392/SO and Hs_ACVRL1_5, SI02659972/SO) and the negative control/SiScramble (1022076) were purchased from Qiagen. We transfected HUVECs when 70% confluent with 20 nM of siRNA. Experimentally, lipofectamine RNAimax (Invitrogen) was mixed with opti-MEM media (Gibco) and incubated at room temperature for 5 min (mix A). Similarly, 20 nM of Alk1 siRNAs (10 nm of Hs_ACVRL1_5 and 10 nm of Hs_ACVRL1_6) or Scramble siRNA were premixed with Opti-MEM for 5 min. Then, mix A and B were combined and incubated for 15 min at room temperature before being added to the cells in EGM™2-Bulletkit™ medium. Cells were used for experiments 72 h post-transfection.

### Western Blot Analysis

Cells were lysed in RIPA buffer (Abcam, ab206996) and equal amounts of proteins were separated on 4-15% gradient Criterion precast gels (Bio-Rad 567-1084). Proteins were then transferred onto nitrocellulose membranes (Bio-Rad). Western Blots were developed with chemiluminescence HRP substrate (Radiance plus, Azure Biosystems AC2103) on a digital image analyzer, Azure Imager c300.

### Reagents and Antibodies

Recombinant proteins: VEGF-A165 (293-VE, R&D Systems) and BMP9 (3209-BP-010). IsolectinB4 labeling reagent (121411). Antibodies: anti-p44/42 MAP kinase (1/1000, phospho-ERK, 9106, Cell Signaling), anti-p44/42 MAP kinase (1/1000, total ERK, 9102, Cell Signaling), anti-ENDOMUCIN (1/200, HM1108, Hycult), anti-aSMA-CY3 (1/200, C6198, Sigma), anti-ENDOGLIN-PE (1/100, 12-1051-82, Thermo-scientific), anti-phospho-AKT (Ser473) (1/500, 4060T, Cell Signaling), anti-AKT (1/500, 9272S, Cell Signaling), anti-JAGGED1 (1/100, AF599-SP, R&D systems), anti-GM130 (1/500, 610823, BD), anti-ERG (1/100, ab92513, Abcam).

### Glycolytic and Mitochondrial Stress Test

HUVECs (70% confluent) were treated with Palbociclib (3 mg/ml) for 12 h in EGM™2-Bulletkit™ medium (CC-3156 & CC-4176, Lonza) prior to seeding onto a Seahorse 24-well tissue culture plate (Agilent Technologies) in EMB-2 media (CC-3162 Lonza) supplemented with 0.1% FBS and Palbociclib (3 mg/ml). To measure respiratory capacity, cells were subjected to a mitochondrial stress test (MST). At the beginning of the assay, the media was changed to DMEM with pyruvate (Thermo-Fisher, pH=7.35 at 37° C.), and the cells were allowed to equilibrate for 30 minutes. Oxygen consumption rate (OCR) was measured using a Seahorse XF24 Flux Analyzer (Agilent Technologies). After three basal OCR measurements (3 min mix, 3 min wait, 4 min measurement), compounds to modulate cellular respiratory function [1 μM Oligomycin (Sigma-Aldrich); 2 μM BAM 15 (Cayman Chemical Company); 1 μM Antimycin A & 100 nM Rotenone (Sigma-Aldrich)] were individually injected after every set of three measurements. Basal respiration was calculated by subtracting the average of the first three measurements by the average of the post-Antimycin A & Rotenone measurements. Respiratory capacity was calculated by subtracting the average of the post-BAM15 measurements by the average of the post-Antimycin A & Rotenone measurements. Reserve capacity was calculated by subtracting the average of the basal measurements from the average of the post-BAM 15 measurements.

### Data Analysis and Statistics.

The Mann-Whitney non-parametric test for unpaired samples was used to analyze continuous variables between groups (GrapPad). One-way ANOVA with Holm-Šidàk's multiple comparison test was used to compare the means of continuous variables among 3 groups. The Two-way ANOVA parametric test for univariate or repeated measures was used to compare two or more groups to determine whether there is an interactive effect between two independent variables (control samples vs. treated samples) on a continuous dependent variable (polarity or time) (IBM SPSS Statistics). All continuous variables were represented as mean±SEM. p<0.05 was considered statistically significant. All graphs and other analyses were generated using Prism 8.0 software (GraphPad). Vessel diameter was calculated using a custom-written MATLAB code (version R2022a; MathWorks). FIG. 3F and FIG. 5C were created with BioRender.com.

### Example 2

Previous work initially focused on Hereditary Hemorrhagic Telangiectasia (HHT), which is a rare autosomal-dominant vascular disease characterized by telangiectasia and larger vascular malformations, which affects 1 in 5,000 children and adults worldwide. HHT causes abnormal connections to develop between arteries and veins, known as arteriovenous malformations (AVMs). The known HHT-causing mutations affect genes encoding the BMP9/10 signaling pathway, namely: ENG, encoding the membrane glycoprotein ENDOGLIN in HHT type 1 and ACVRL1, encoding the membrane receptor ALK1 in HHT type 2. The most common organs affected by vascular malformations in HHT patients are the nose, skin, lungs, brain, and liver. In the nose and skin, the telangiectasias are prone to rupture and cause uncontrolled nose bleeding that can ultimately result in chronic anemia. In the lungs, brain and liver, AVMs can lead to life-threatening pulmonary hemorrhage, stroke or liver failure. Symptoms often begin during childhood and progress in severity. However, most patients typically do not receive a definitive HHT diagnosis until >40 years of age since they do not display any warning signs before AVMs rupture and/or do not receive genetic counseling or testing for HHT. To date, there are no FDA-approved drugs for HHT, and current treatments are unsatisfactory, as they involve invasive surgery or radiation, with considerable risks and undesirable side-effects.

Vascular endothelial cells (ECs) lining blood vessels are the primary cells affected in HHT. In ECs, decreased activity of BMP9/10 signaling leads to over-activation of proangiogenic factors, namely, VEGF-A and angiopoietin, triggering EC hyperproliferation, as well as alterations in their permeability ultimately leading to AVMs. Cell proliferation is strictly regulated by cell cycle control via several molecular checkpoints. While aberrant EC proliferation has been identified in AVMs in preclinical models of HHT, as well as in HHT patients, the link between endothelial cell cycle dysregulation and the development of AVMs has not yet been investigated.

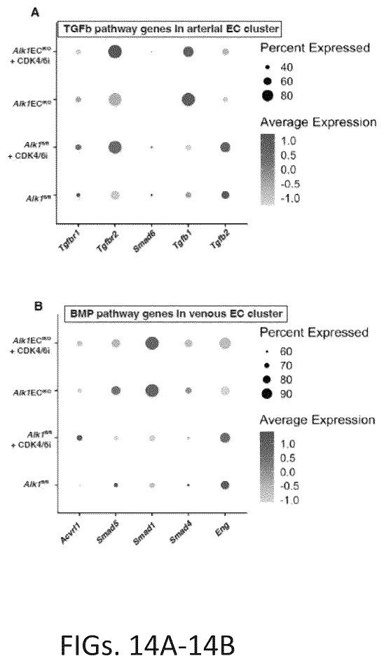

This example discusses that endothelial cell cycle dysregulation is a mechanism involved in the development of arterio-venous malformations in HHT, as well as other types of vascular malformations named above. Palbociclib, a FDA-approved cell cycle inhibitor, could be used to prevent arterio-venous and other malformations, including venous, capillary, lymphatic and cerebral cavernous malformations. In addition, data is provided that shows that, similar to Palbociclib, CVT-313 and Ribociclib inhibit EC proliferation in vitro, as assessed by EdU uptake (FIG. 16).

### Prevention-Regression:

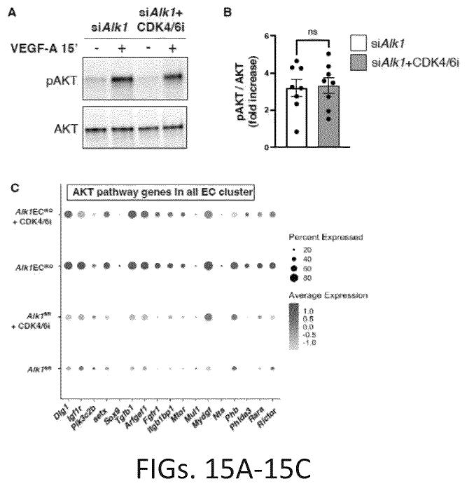

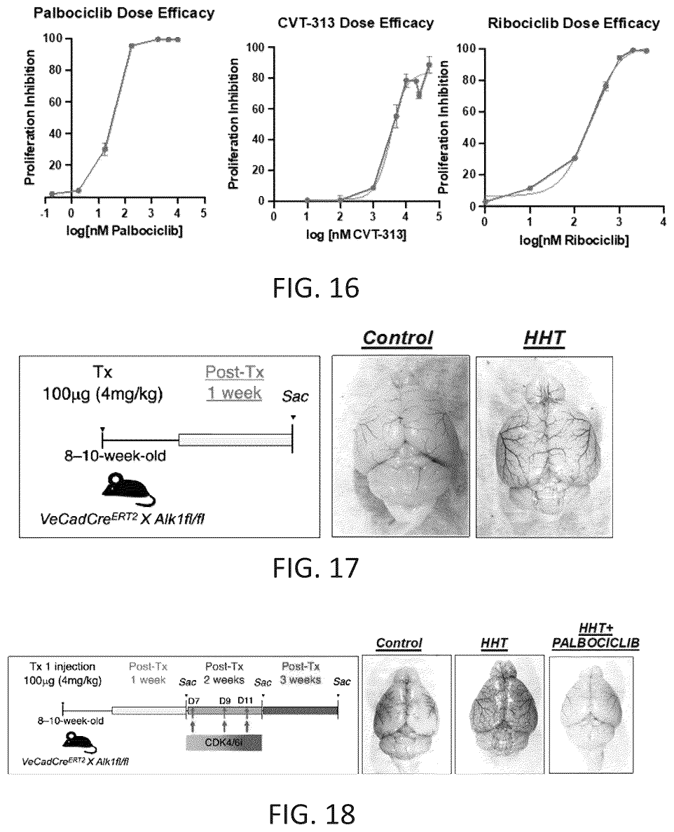

We have demonstrated that Palbociclib prevented the development of arterio-venous malformations in preclinically a model of HHT (Alk1 mutant mice) at a juvenile stage of postnatal development. Additional data showed that at adult stages, Palbociclib regressed anomalies observed in the brain vasculature of Alk1 mutant mice (FIGS. 17 and 18). Palbociclib, and other cell cycle modulators such as CVT-313 and Ribociclib, could be used to regress arterio-venous malformations in HHT and other types of vascular malformations.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1 percent to about 5 percent” should be interpreted to include not only the explicitly recited concentration of about 0.1 weight percent to about 5 weight percent but also include individual concentrations (e.g., 1 percent, 2 percent, 3 percent, and 4 percent) and the sub-ranges (e.g., 0.5 percent, 1.1 percent, 2.2 percent, 3.3 percent, and 4.4 percent) within the indicated range. The term “about” can include traditional rounding according to significant figures of the numerical value. In addition, the phrase “about ‘x’ to ‘y’” includes “about ‘x’ to about ‘y’”.

Many variations and modifications may be made to the above-described aspects. All such modifications and variations are intended to be included herein within the scope of this disclosure and protected by the following claims.

What is claimed:

1. A method for treating a vascular malformation, comprising: administering to a subject in need thereof, a pharmaceutical composition, wherein the pharmaceutical composition comprises a therapeutically effective amount of the pharmaceutical composition, wherein the pharmaceutical composition includes: palbociclib or pharmaceutically acceptable salts thereof; ribociclib or pharmaceutically acceptable salts thereof; CVT-313 or pharmaceutically acceptable salts thereof; or abemaciclib, or pharmaceutically acceptable salts thereof.

2. The method of claim 1, wherein the subject is an adult or a child, wherein treating comprises treating the adult or child that has developed the vascular malformation.

3. The method of claim 2, wherein the vascular malformation is arterio-venous malformations, cerebral cavernous malformations, venous malformations, or lymphatic malformations.

4. The method of claim 1, wherein the subject is a neonate, wherein the treating comprises prophylactically treating the neonate.

5. The method of claim 4, wherein the neonate has been diagnosed to develop the vascular malformation.

6. The method of claim 5, wherein the vascular malformation is arterio-venous malformations, cerebral cavernous malformations, venous malformations, or lymphatic malformations.

7. The method of claim 1, wherein the pharmaceutical composition includes palbociclib or pharmaceutically acceptable salts thereof.

8. The method of claim 1, wherein the pharmaceutical composition includes ribociclib or pharmaceutically acceptable salts thereof.

9. The method of claim 1, wherein the pharmaceutical composition includes CVT-313 or pharmaceutically acceptable salts thereof.

10. The method of claim 1, wherein the pharmaceutical composition includes abemaciclib, or pharmaceutically acceptable salts thereof.

11. A method to prevent or treat endothelial cell hyperproliferation comprising administering to a subject in need thereof a pharmaceutical composition, wherein the pharmaceutical composition comprises a therapeutically effective amount of the pharmaceutical composition, wherein the pharmaceutical composition includes: palbociclib or pharmaceutically acceptable salts thereof; a ribociclib or pharmaceutically acceptable salts thereof; a CVT-313 or pharmaceutically acceptable salts thereof; or abemaciclib, or pharmaceutically acceptable salts thereof.

12. The method of claim 11, wherein the subject is an adult or a child, wherein treating comprises treating the adult or the child that has developed endothelial cell hyperproliferation.

13. The method of claim 11, wherein the subject is a neonate, wherein the treating comprises prophylactically treating the neonate.

14. The method of claim 11, wherein the pharmaceutical composition includes ribociclib or pharmaceutically acceptable salts thereof.

15. The method of claim 11, wherein the pharmaceutical composition includes CVT-313 or pharmaceutically acceptable salts thereof.

16. The method of claim 11, wherein the pharmaceutical composition includes abemaciclib, or pharmaceutically acceptable salts thereof.

17. The method of claim 11, wherein the pharmaceutical composition includes palbociclib or pharmaceutically acceptable salts thereof.
